(19) United States
(12) Patent Application Publication
(10) Pub. No.: US 2002/0128267 A1
(43) Pub. Date: Sep. 12, 2002

(54) Method of using COX-2 inhibitors in the treatment and prevention of ocular COX-2 mediated disorders

(76) Inventors: Rebanta Bandyopadhyay, Portage, MI (US); David Eveleth, East Brunswick, NJ (US); Thomas Joseph Van Haarlem, Clinton, NJ (US); Tugrul T. Kararli, Skokie, IL (US); Satish K. Singh, Portage, MI (US)

Correspondence Address: Pharmacia Corporation, Corporate Patent Department, P.O. Box 5110, Chicago, IL, 60680-9889, US

(21) Appl. No.: 09/849,683

(22) Filed: May 4, 2001

Related U.S. Application Data
(60) Provisional application No. 60/218,101, filed on Jul. 13, 2000.
(60) Provisional application No. 60/279,285, filed on Mar. 28, 2001.

(51) Int. Cl. A61K 31/415; A61K 31/50; A61K 31/12; A61K 31/353

(52) U.S. Cl. 514/247; 514/406; 514/456; 514/471; 514/684

(57) ABSTRACT

The present invention provides methods for the treatment and prevention of ocular COX-2 mediated disorders using COX-2 inhibitors.

This application claims the benefit of priority of U.S. Provisional Application Ser. No. 60/218101, filed Jul. 13, 2000 and of U.S. Provisional Application Ser. No. 60/279285, filed Mar. 28, 2001.

### FIELD OF THE INVENTION

The present invention relates to methods for the treatment and prevention of ocular COX-2 mediated disorders using COX-2 inhibitors.

### BACKGROUND OF THE INVENTION

Prostaglandins play a major role in the inflammation process and the inhibition of prostaglandin production, especially production of PGG2, PGH2 and PGE2, has been a common target of anti-inflammatory drug discovery. However, common non-steroidal anti-inflammatory drugs (NSAIDs) that are active in reducing the prostaglandin-induced pain and swelling associated with the inflammation process are also active in affecting other prostaglandin-regulated processes not associated with the inflammation process. Thus, use of high doses of most common NSAIDs can produce severe side effects, including life-threatening ulcers, which limit their therapeutic potential. An alternative to NSAIDs is the use of corticosteroids, which have even more drastic side effects, especially when long-term therapy is involved.

Previous NSAIDs have been found to prevent the production of prostaglandins by inhibiting enzymes in the human arachidonic acid/prostaglandin pathway, including the enzyme cyclooxygenase (COX). The recent discovery of an inducible enzyme associated with inflammation (named “cyclooxygenase-2 (COX-2)” or “prostaglandin G/H synthase II”) provides a viable target of inhibition that more effectively reduces inflammation and produces fewer and less drastic side effects.

Numerous compounds have been reported having therapeutically or prophylactically useful selective COX-2 inhibitory effect, and have been disclosed as having utility in treatment or prevention of specific COX-2 mediated disorders or of such disorders in general. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib, and the compound 4-[5-(3-fluoro-4-methoxyphenyl)-3-difluoromethyl)-1H-pyrazol-1-yl]benzenesulfonamide, also referred to herein as deracoxib. Celecoxib has the structure shown in formula (I):

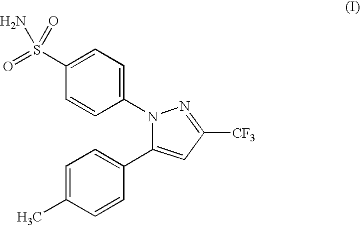

and deracoxib has the structure shown in formula (II):

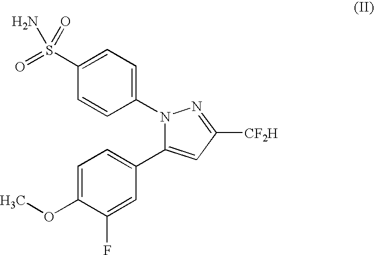

Other compounds reported to have therapeutically or prophylactically useful selective COX-2 inhibitory effect are substituted isoxazolyl benzenesulfonamides as reported in U.S. Pat. No. 5,633,272 to Talley et al., including for example the compound 4-[5-methyl-3-phenylisoxazol-4-yl]-benzenesulfonamide, also referred to herein as valdecoxib, which has the structure shown in formula (III):

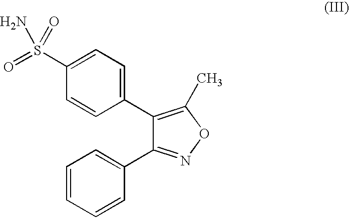

Parecoxib, disclosed in U.S. Pat. No. 5,932,598 to Talley et al., is one of a class of water-soluble prodrugs of selective COX-2 inhibitory drugs. Parecoxib, which has the structure shown in formula (IV) below, rapidly converts to the substantially water-insoluble selective COX-2 inhibitory drug valdecoxib following administration to a subject.

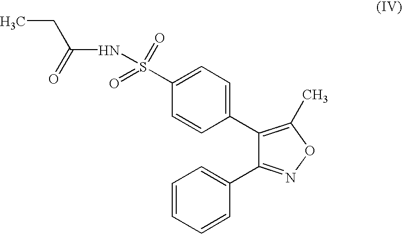

Still other compounds reported to have therapeutically or prophylactically useful selective COX-2 inhibitory effect are substituted (methylsulfonyl)-phenyl furanones as reported in U.S. Pat. No. 5,474,995 to Ducharme et al., including for example the compound 3-phenyl-4-[4-(methylsulfonyl)phenyl]-5H-furan-2-one, also referred to herein as rofecoxib, which has the structure shown in formula (V):

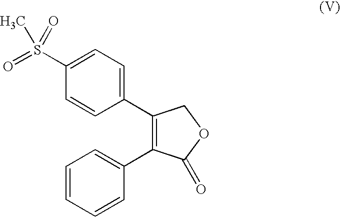

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine, also referred to herein as etoricoxib, which has the structure shown in formula (VI):

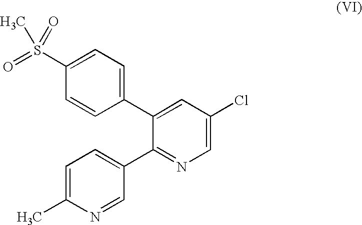

Europena Patent Application No. 0 863 134 discloses the compound 2-(3,5-difluorophenyl)-3-[4-(methylsulfonyl) phenyl] -2-cyclopenten-1-one said to be, useful as a selective COX-2 inhibitory drug.

International Patent Publication No. WO 00/24719 discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

The various classes of compounds that are selective inhibitors of COX-2 have been reviewed by J. Talley in Prog. Med. Chem., 36, 201-234 (1999). Compounds that selectively inhibit COX-2 have also been described in the following individual publications.

WO 96/06840.

WO 96/03388.

WO 96/03387.

WO 96/25405.

WO 95/15316.

WO 94/27980.

WO 95/00501.

WO 94/13635.

WO 94/20480.

WO 94/26731.

Inflammation is associated with a variety of ocular disorders. Inflammation may also result from a number of ophthalmic surgical procedures, including cataract surgery (“Postsurgery Intraocular Inflammation,” K. Schmitz, et al., Developments in Ophthalmology, 31, 175-191 (1999)). Corticosteroids are used frequently as ocular anti-inflammatory agents, however, they increase the risk of glaucoma by raising the intraocular pressure (IOP) when administered exogenously (R. C. Tripathi, et al., Drugs and Aging, 15(6), 439-450 (1999)).

Non-steroidal anti-inflammatory agents (NSAIDs) have been used to treat ocular inflammation (N. Samiy, et al., International Ophthalmology Clinics, 36(1), 195-206 (1996)). WO 95/31968 discloses the use of NSAIDs and particularly diclofenac sodium for treating inflammatory diseases of the eye. WO 99/59634 teaches the use of the selective COX-2 inhibitors, etodolac, NS-398 and meloxicam as anti-inflammatory eye-drops. Recent work suggests that the production of inflammatory amounts of prostaglandins in ocular tissues is the result of COX-2 expression, while the normal production of prostaglandins in the eye is the result of constitutively expressed COX-1 (J. L. Masferrer, et al., Survey of Ophthalmology, 41(Supp. 2), S35-S40 (1997)).

The addition of the NSAID, diclofenac sodium, to latanoprost therapy, is reported to prevent the disruption of the blood-aqueous barrier and decrease the incidence of angiographic cystoid macular edema in early postoperative pseudophakias (K. Miyake, et al., Arch Ophthalmol, (117), 34-40 (1999)).

WO 96/38442 discloses substituted sulfonylphenylheterocyclyl COX-2 inhibitors having utility in the treatment of sarcoidosis and conjunctivitis.

Masferrer & Kulkarni (1997), in Survey of Ophthalmology, 41(Supp. 2), S35-S40, further teach utility of selective COX-2 inhibitory drugs in treating ocular pain and postoperative ocular inflammation.

EP 995747 discloses certain substituted sulfonylphenylheterocyclyl COX-2 inhibitors useful for the treatment of post-ophthalmic surgery inflammation, including from cataract and refractive surgery. It is further disclosed that the subject COX-2 inhibitors are useful in the treatment of ophthalmic diseases such as sarcoidosis, retinitis, retinopathies, uveitis, ocular photophobia and acute injury to the eye tissue.

U.S. Pat. No. 5,466,823 discloses pyrazolyl COX-2 inhibitors useful in the treatment of inflammation and inflammation-related disorders, including sarcoidosis and conjunctivitis.

U.S. Pat. No. 5,521,207 discloses pyrazolyl COX-2 inhibitors useful in the treatment of inflammation and inflammation-related disorders, including sarcoidosis and conjunctivitis.

EP 0924201 discloses tricyclic-substituted pyrazolyl benzenesulfonamides as COX-2 inhibitors useful in the treatment of sarcoidosis and conjunctivitis.

WO 00/32189 discloses orally deliverable compositions of celecoxib, a pyrazole COX-2 inhibitor, useful in the treatment of post-operative inflammation including from ophthalmic surgery such as cataract surgery and refractive surgery, and treatment of ophthalmic diseases, such as retinitis, retinopathies, conjunctivitis, uveitis, ocular photophobia, acute injury to the eye tissue, glaucoma and sarcoidosis.

WO 00/66562 discloses pyrazolyl COX-2 inhibitors useful in the treatment of post-ophthalmic surgery inflammation, such as cataract surgery and refractive surgery and in the treatment of ophthalmic diseases, such as retinitis, retinopathies, conjunctivitis, uveitis, ocular photophobia, acute injury to the eye tissue, glaucoma and sarcoidosis.

WO 99/64415 discloses the preparation of heterocyclyl sulfonylbenzene compounds, including pyrazoles, having utility in the treatment of diabetic retinopathy.

WO 96/3385 discloses 3,4-substituted pyrazoles as COX-2 inhibitors useful in the treatment of sarcoidosis and conjunctivitis.

WO 97/13755 discloses 1,3,5-trisubstituted pyrazole COX-2 inhibitors useful in the treatment of inflammatory conditions, including inflammatory eye conditions, such as conjunctivitis, uveitis and sarcoidosis.

WO 99/15505 discloses 1,5-diphenyl pyrazole COX-2 inhibitors useful in the treatment of inflammatory conditions, including inflammatory eye conditions, such as conjunctivitis, uveitis and sarcoidosis.

WO 99/25695 discloses 5-aryl pyrazole COX-2 inhibitors useful in the treatment of inflammatory conditions, including inflammatory eye conditions, such as conjunctivitis, uveitis and sarcoidosis.

U.S. Pat. No. 5,633,272 discloses isoxazolyl COX-2 inhibitors useful in the treatment of inflammation and inflammation-related disorders, including sarcoidosis and conjunctivitis.

WO 98/06708 discloses a stable crystalline form of valdecoxib, an isoxazolyl COX-2 inhibitor, which is useful in the treatment of post-ophthalmic surgery inflammation and in the treatment of ophthalmic diseases.

WO 00/23433 discloses benzopyran COX-2 inhibitors useful in the treatment of post-ophthalmic surgery inflammation, such as cataract surgery and refractive surgery and in the treatment of ophthalmic diseases, such as retinitis, retinopathies, conjunctivitis, uveitis, ocular photophobia, acute injury to the eye tissue, glaucoma and sarcoidosis. It is further disclosed that the benzopyran COX-2 inhibitors have utility in the treatment of ophthalmological conditions such as corneal graft rejection, ocular neovascularization, retinal neovascularization including neovascularization following injury or infection, diabetic retinopathy, macular degeneration, retrolental fibroplasia and neovascular glaucoma.

WO 98/47890 discloses benzopyran COX-2 inhibitors useful in the treatment of post-ophthalmic surgery inflammation, such as cataract surgery and refractive surgery and in the treatment of ophthalmic diseases, such as retinitis, retinopathies, conjunctivitis, uveitis, ocular photophobia, acute injury to the eye tissue, glaucoma and sarcoidosis. It is further disclosed that the benzopyran COX-2 inhibitors have utility in the treatment of ophthalmological conditions such as corneal graft rejection, ocular neovascularization, retinal neovascularization including neovascularization following injury or infection, diabetic retinopathy, macular degeneration, retrolental fibroplasia and neovascular glaucoma.

WO 00/10993 discloses diarylbenzopyran derivatives as COX-2 inhibitors useful in the treatment of diabetic retinopathy and glaucoma.

U.S. Pat. No. 5,932,598 discloses prodrugs of COX-2 inhibitors useful in the treatment of inflammation and inflammation-related disorders, including post-operative inflammation from ophthalmic surgery and other ophthalmic diseases, such as retinitis, retinopathies, uveitis, ocular photophobia and acute injury to the eye.

WO 96/19469 discloses diaryl-2-(5H)-furnanones as COX-2 inhibitors useful in the treatment of diabetic retinopathy.

WO 99/23087 discloses diaryl-5-alkyl-5-methyl-2(5H)-furanones as COX-2 inhibitors useful in the treatment of glaucoma.

WO 99/15513 discloses a process for making 3-aryloxy-4-arylfuran-2-ones, which are COX-2 inhibitors useful in the treatment of glaucoma.

WO 98/41516 discloses (methylsulfonyl)phenyl-2-(5H)-furanones with an oxygen link as COX-2 inhibitors, useful in the treatment of diabetic retinopathy and glaucoma.

WO 97/16435 discloses 3,4-diaryl-2-hydroxy-2,5-dihydrofurans as prodrugs to COX-2 inhibitors having utility in the treatment of diabetic retinopathy and glaucoma.

WO 97/14691 discloses (methylsulfonyl)phenyl-2-(5H)-furanones as COX-2 inhibitors useful in the treatment of diabetic retinopathy and glaucoma.

WO 96/36623 discloses diaryl-5-oxygenated-2-(5H)-furanones as COX-2 inhibitors having utility in the treatment of diabetic retinopathy.

WO 98/03484 discloses substituted pyridines as selective COX-2 inhibitors useful in the treatment of glaucoma.

WO 96/24585 discloses 3,4-diaryl substituted pyridinyl COX-2 inhibitors useful for the treatment of inflammation, including sarcoidosis and conjunctivitis.

WO 96/24584 discloses 2,3-substituted pyridinyl COX-2 inhibitors useful for the treatment of inflammation, including sarcoidosis and ophthalmic diseases such as retinitis, retinopathies, uveitis, conjunctivitis, and acute injury to the eye tissue.

U.S. Pat. No. 5,916,905 discloses 2,3-diarylpyridinyl COX-2 inhibitors having utility in the treatment of sarcoidosis and ophthalmic diseases such as retinitis, retinopathies, uveitis, conjunctivitis, and acute injury to the eye tissue.

WO 99/14195 discloses 2-aminopyridines as COX-2 inhibitors having utility in the treatment of diabetic retinopathy and glaucoma.

WO 99/14194 discloses 2,3,5-trisubstituted pyridines as inhibitors of COX-2, useful in the treatment of diabetic retinopathy and glaucoma.

WO 96/31509 discloses imidazo[1,2-a]pyridine COX-2 inhibitors useful in the treatment of inflammatory disorders, including ophthalmic diseases.

WO 00/26216 discloses pyrazolo[l,5-a]pyridine COX-2 inhibitors useful in the treatment of inflammatory disorders, including ophthalmic diseases.

EP 863134 discloses the preparation of 2-(3,5-difluorophenyl)-3-[4-(methylsulfonyl)phenyl]-2-cyclopenten-1-one useful as a selective inhibitor of COX-2 in the treatment of diabetic retinopathy and glaucoma.

WO 98/41511 discloses pyridazinones as COX-2 inhibitors having utility in the treatment of diabetic retinopathy and glaucoma.

International Patent Publication No. WO 00/25771, incorporated herein by reference, discloses ophthalmic compositions comprising a prostaglandin analog such as latanoprost and an anti-inflammatory agent.

### SUMMARY OF THE INVENTION

A need therefore remains for a method of treating or preventing COX-2 mediated disorders of the eye. A special need exists for such a method having its therapeutic or prophylactic effect through selective inhibition of cyclooxygenase-2 (COX-2), without the undesirable side-effects associated with inhibition of cyclooxygenase-1 (COX-1) that can occur with conventional NSAIDS. There is a continued need for more efficacious and safer therapeutic agents for the prevention and treatment of a variety of ocular COX-2 mediated disorders.

To address the continuing need to find safe and effective agents for the prophylaxis and treatment of ocular COX-2 mediated disorders, therapeutic combinations and methods are now reported.

Among its several embodiments, the present invention provides a therapeutic method for treating or preventing an ocular COX-2 mediated disorder comprising administering a source of a COX-2 inhibitor compound to a mammal in need of such treatment, where the disorder is selected from blepharitis, post-operative inflammation and pain from corneal transplant surgery, endophthalmitis, episcleritis, keratitis, keratoconjunctivitis, keratoconjunctivitis sicca, post-operative inflammation and pain from lens implantation surgery, Mooren's ulcer, and post-operative inflammation and pain from retinal detachment surgery. Preferred COX-2 inhibitors are celecoxib, deracoxib, valdecoxib, a benzopyran COX-2 inhibitor, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methylbutoxy)-5-[4-(methylsulfonyl)phenyl]-3(2H)-pyridazinone.

In another embodiment, the source of the COX-2 inhibitor compound is a prodrug of a COX-2 inhibitor compound, illustrated herein with parecoxib.

In yet another embodiment, the invention provides a therapeutic method for treating or preventing an ocular COX-2 mediated disorder comprising administering celecoxib to a mammal in need of such treatment, where the disorder is selected from macular edema, intraoperative miosis and ocular pain.

In still another embodiment, the invention provides a therapeutic method for treating or preventing an ocular COX-2 mediated disorder comprising administering deracoxib to a mammal in need of such treatment, where the disorder is selected from post-operative inflammation and pain from cataract surgery, acute injury to the eye tissue, glaucoma, macular edema, intraoperative miosis, ocular pain, photophobia, post-operative inflammation and pain from refractive surgery, retinitis, retinopathies and uveitis.

In another embodiment, the invention provides a therapeutic method for treating or preventing an ocular COX-2 mediated disorder comprising administering valdecoxib to a mammal in need of such treatment, where the disorder is selected from macular edema, intraoperative miosis and ocular pain.

In yet another embodiment, the invention provides a therapeutic method for treating or preventing an ocular COX-2 mediated disorder comprising administering a benzopyran COX-2 inhibitor to a mammal in need of such treatment, where the disorder is selected from glaucoma, macular edema, intraoperative miosis and ocular pain.

In still another embodiment, the invention provides a therapeutic method for treating or preventing an ocular COX-2 mediated disorder comprising administering parecoxib to a mammal in need of such treatment, where the disorder is selected from conjunctivitis, glaucoma, macular edema, intraoperative miosis and ocular pain.

In another embodiment, the invention provides a therapeutic method for treating or preventing an ocular COX-2 mediated disorder comprising administering rofecoxib to a mammal in need of such treatment, where the disorder is selected from post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, sarcoidosis and uveitis.

In yet another embodiment, the invention provides a therapeutic method for treating or preventing an ocular COX-2 mediated disorder comprising administering etoricoxib to a mammal in need of such treatment, where the disorder is selected from post-operative inflammation and pain from cataract surgery, conjunctivitis, acute injury to the eye tissue, macular edema, intraoperative miosis, ocular pain, photophobia, post-operative inflammation and pain from refractive surgery, retinitis, retinopathies, sarcoidosis and uveitis.

In still another embodiment, the invention provides a therapeutic method for treating or preventing an ocular COX-2 mediated disorder comprising administering 2-(3,5-difluorophenyl)-3-[4-(methylsulfonyl)phenyl]-2-cyclopenten-1-one to a mammal in need of such treatment, where the disorder is selected from post-operative inflammation and pain from cataract surgery, conjunctivitis, acute injury to the eye tissue, macular edema, intraoperative miosis, ocular pain, photophobia, post-operative inflammation and pain from refractive surgery, retinitis, sarcoidosis and uveitis.

In another embodiment, the invention provides a therapeutic method for treating or preventing an ocular COX-2 mediated disorder comprising administering 2-(3,4-difluorophenyl)-4-(3-hydroxy-3-methylbutoxy)-5-[4-(methylsulfonyl)phenyl]-3(2H)-pyridazinone to a mammal in need of such treatment, where the disorder is selected from post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, retinopathies, sarcoidosis and uveitis.

In yet another embodiment, the invention provides a pharmaceutical composition for treating or preventing Mooren's ulcer, in a mammal in need of such treatment, consisting essentially of a source of a COX-2 inhibitor compound and one or more ophthalmically acceptable excipient ingredients that reduce the rate of removal of the composition from the eye by lacrimation such that the composition has an effective residence time in the eye of about 2 to about 24 hours.

Further scope of the applicability of the present invention will become apparent from the detailed description provided below. However, it should be understood that the following detailed description and examples, while indicating preferred embodiments of the invention, are given by way of illustration only since various changes and modifications within the spirit and scope of the invention will become apparent to those skilled in the art from this detailed description.

### DETAILED DESCRIPTION OF THE INVENTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

The contents of each of the references cited herein, including the contents of the references cited within these primary references, are herein incorporated by reference in their entirety.

Definitions

The following definitions are provided in order to aid the reader in understanding the detailed description of the present invention.

The phrase “cyclooxygenase-2 inhibitor” or “COX-2 inhibitor” or “cyclooxygenase-II inhibitor” includes agents that specifically inhibit a class of enzymes, cyclooxygenase-2, with less significant inhibition of cyclooxygenase-1.

Preferably, it includes compounds that have a cyclooxygenase-2 IC₅₀ of less than about 0.2 μM, and also have a selectivity ratio of cyclooxygenase-2 inhibition over cyclooxygenase-1 inhibition of at least 50, and more preferably of at least 100. Even more preferably, the compounds have a cyclooxygenase-1 IC₅₀ of greater than about 1 μM, and more preferably of greater than 10 μM.

The phrase “combination therapy” (or “co-therapy”) embraces the administration of a COX-2 inhibitor and another therapeutic agent as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” generally is not intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies.

The phrase “therapeutically effective” is intended to qualify the amount of inhibitors in the therapy. This amount will achieve the goal of reducing or eliminating ocular inflammation.

“Therapeutic compound” means a compound useful in the prophylaxis or treatment of ocular inflammation.

The term “comprising” means “including the following elements but not excluding others.”

The term “hydrido” denotes a single hydrogen atom (H). This hydrido radical may be attached, for example, to an oxygen atom to form a hydroxyl radical or two hydrido radicals may be attached to a carbon atom to form a methylene (—CH₂—) radical. Where used, either alone or within other terms such as “haloalkyl”, “alkylsulfonyl”, “alkoxyalkyl” and “hydroxyalkyl”, the term “alkyl” embraces linear or branched radicals having one to about twenty carbon atoms or, preferably, one to about twelve carbon atoms. More preferred alkyl radicals are “lower alkyl” radicals having one to about ten carbon atoms. Most preferred are lower alkyl radicals having one to about six carbon atoms. Examples of such radicals include methyl, ethyl, n-propyl, isopropyl, n-butyl, isobutyl, sec-butyl, tert-butyl, pentyl, iso-amyl, hexyl and the like.

The term “alkenyl” embraces linear or branched radicals having at least one carbon-carbon double bond of two to about twenty carbon atoms or, preferably, two to about twelve carbon atoms. More preferred alkenyl radicals are “lower alkenyl” radicals having two to about six carbon atoms. Examples of alkenyl radicals include ethenyl, propenyl, allyl, propenyl, butenyl and 4-methylbutenyl.

The term “alkynyl” denotes linear or branched radicals having two to about twenty carbon atoms or, preferably, two to about twelve carbon atoms. More preferred alkynyl radicals are “lower alkynyl” radicals having two to about ten carbon atoms. Most preferred are lower alkynyl radicals having two to about six carbon atoms. Examples of such radicals include propargyl, butynyl, and the like.

The terms “alkenyl”, “lower alkenyl”, embrace radicals having “cis” and “trans” orientations, or alternatively, “E” and “Z” orientations.

The term “cycloalkyl” embraces saturated carbocyclic radicals having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl. The term “cycloalkenyl” embraces partially unsaturated carbocyclic radicals having three to twelve carbon atoms. More preferred cycloalkenyl radicals are “lower cycloalkenyl” radicals having four to about eight carbon atoms. Examples of such radicals include cyclobutenyl, cyclopentenyl, cyclopentadienyl and cyclohexenyl.

The term “halo” means halogens such as fluorine, chlorine, bromine or iodine. The term “haloalkyl” embraces radicals wherein any one or more of the alkyl carbon atoms is substituted with halo as defined above. Specifically embraced are monohaloalkyl, dihaloalkyl and polyhaloalkyl radicals. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having one to six carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl.

The terms “alkoxy” and “alkyloxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. The term “alkoxyalkyl” embraces alkyl radicals having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one, two or three rings wherein such rings may be attached together in a pendent manner or may be fused. The term “aryl” embraces aromatic radicals such as phenyl, naphthyl, tetrahydronaphthyl, indane and biphenyl. Aryl moieties may also be substituted at a substitutable position with one or more substituents selected independently from alkyl, alkoxyalkyl, alkylaminoalkyl, carboxyalkyl, alkoxycarbonylalkyl, aminocarbonylalkyl, alkoxy, aralkoxy, hydroxyl, amino, halo, nitro, alkylamino, acyl, cyano, carboxy, aminocarbonyl, alkoxycarbonyl and aralkoxycarbonyl.

The term “heterocyclo” embraces saturated, partially unsaturated and unsaturated heteroatom-containing ring-shaped radicals, where the heteroatoms may be selected from nitrogen, sulfur and oxygen. Examples of saturated heterocyclo radicals include saturated 3 to 6-membered heteromonocyclic groups containing 1 to 4 nitrogen atoms (e.g. pyrrolidinyl, imidazolidinyl, piperidino, piperazinyl, etc.); saturated 3 to 6-membered heteromonocyclic group containing 1 to 2 oxygen atoms and 1 to 3 nitrogen atoms (e.g. morpholinyl, etc.); saturated 3 to 6-membered heteromonocyclic group containing 1 to 2 sulfur atoms and 1 to 3 nitrogen atoms (e.g., thiazolidinyl, etc.). Examples of partially unsaturated heterocyclo radicals include dihydrothiophene, dihydropyran, dihydrofuran and dihydrothiazole.

The term “heteroaryl” embraces unsaturated heterocyclo radicals. Examples of unsaturated heterocyclo radicals, also termed “heteroaryl” radicals include unsaturated 3 to 6 membered heteromonocyclic group containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.) tetrazolyl (e.g. 1H-tetrazolyl, 2H-tetrazolyl, etc.), etc.; unsaturated condensed heterocyclo group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic group containing an oxygen atom, for example, pyranyl, furyl, etc.; unsaturated 3 to 6-membered heteromonocyclic group containing a sulfur atom, for example, thienyl, etc.; unsaturated 3- to 6-membered heteromonocyclic group containing 1 to 2 oxygen atoms and 1 to 3 nitrogen atoms, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo group containing 1 to 2 oxygen atoms and 1 to 3 nitrogen atoms (e.g. benzoxazolyl, benzoxadiazolyl, etc.); unsaturated 3 to 6-membered heteromonocyclic: group containing 1 to 2 sulfur atoms and 1 to 3 nitrogen atoms, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo group containing 1 to 2 sulfur atoms and 1 to 3 nitrogen atoms (e.g., benzothiazolyl, benzothiadiazolyl, etc.) and the like. The term also embraces radicals where heterocyclo radicals are fused with aryl radicals. Examples of such fused bicyclic radicals include benzofuran, benzothiophene, benzopyran, and the like. The terms benzopyran and chromene are interchangeable. Said “heterocyclo group” may have 1 to 3 substituents such as alkyl, hydroxyl, halo, alkoxy, oxo, amino and alkylamino.

The term “alkylthio” embraces radicals containing a linear or branched alkyl radical, of one to about ten carbon atoms attached to a divalent sulfur atom. More preferred alkylthio radicals are “lower alkylthio” radicals having alkyl radicals of one to six carbon atoms. Examples of such lower alkylthio radicals are methylthio, ethylthio, propylthio, butylthio and hexylthio. The term “alkylthioalkyl” embraces radicals containing an alkylthio radical attached through the divalent sulfur atom to an alkyl radical of one to about ten carbon atoms. More preferred alkylthioalkyl radicals are “lower alkylthioalkyl” radicals having alkyl radicals of one to six carbon atoms. Examples of such lower alkylthioalkyl radicals include methylthiomethyl.

The term “alkylsulfinyl” embraces radicals containing a linear or branched alkyl radical, of one to ten carbon atoms, attached to a divalent —S(═O)— radical. More preferred alkylsulfinyl radicals are “lower alkylsulfinyl” radicals having alkyl radicals of one to six carbon atoms. Examples of such lower alkylsulfinyl radicals include methylsulfinyl, ethylsulfinyl, butylsulfinyl and hexylsulfinyl.

The term “sulfonyl”, whether used alone or linked to other terms such as alkylsulfonyl, denotes respectively divalent radicals —SO₂—. “Alkylsulfonyl” embraces alkyl radicals attached to a sulfonyl radical, where alkyl is defined as above. More preferred alkylsulfonyl radicals are “lower alkylsulfonyl” radicals having one to six carbon atoms. Examples of such lower alkylsulfonyl radicals include methylsulfonyl, ethylsulfonyl and propylsulfonyl. The “alkylsulfonyl” radicals may be further substituted with one or more halo atoms, such as fluoro, chloro or bromo, to provide haloalkylsulfonyl radicals.

The terms “sulfamyl”, “aminosulfonyl” and “sulfofamidyl” denote NH₂O₂S—.

The term “acyl” denotes a radical provided by the residue after removal of hydroxyl from an organic acid. Examples of such acyl radicals include alkanoyl and aroyl radicals. Examples of such lower alkanoyl radicals include formyl, acetyl, propionyl, butyryl, isobutyryl, valeryl, isovaleryl, pivaloyl, hexanoyl, trifluoroacetyl.

The term “carbonyl”, whether used alone or with other terms, such as “alkoxycarbonyl”, denotes —(C═O)—. The term “aroyl” embraces aryl radicals with a carbonyl radical as defined above. Examples of aroyl include benzoyl, naphthoyl, and the like and the aryl in said aroyl may be additionally substituted.

The terms “carboxy” or “carboxyl”, whether used alone or with other terms, such as “carboxyalkyl”, denotes —CO₂H. The term “carboxyalkyl” embraces alkyl radicals substituted with a carboxy radical. More preferred are “lower carboxyalkyl” which embrace lower alkyl radicals as defined above, and may be additionally substituted on the alkyl radical with halo. Examples of such lower carboxyalkyl radicals include carboxymethyl, carboxyethyl and carboxypropyl. The term “alkoxycarbonyl” means a radical containing an alkoxy radical, as defined above, attached via an oxygen atom to a carbonyl radical. More preferred are “lower alkoxycarbonyl” radicals with alkyl portions having 1 to 6 carbons. Examples of such lower alkoxycarbonyl (ester) radicals include substituted or unsubstituted methoxycarbonyl, ethoxycarbonyl, propoxycarbonyl, butoxycarbonyl and hexyloxycarbonyl.

The terms “alkylcarbonyl”, “arylcarbonyl” and “aralkylcarbonyl” include radicals having alkyl, aryl and aralkyl radicals, as defined above, attached to a carbonyl radical. Examples of such radicals include substituted or unsubstituted methylcarbonyl, ethylcarbonyl, phenylcarbonyl and benzylcarbonyl.

The term “aralkyl” embraces aryl-substituted alkyl radicals such as benzyl, diphenylmethyl, triphenylmethyl, phenylethyl, and diphenylethyl. The aryl in said aralkyl may be additionally substituted with halo, alkyl, alkoxy, haloalkyl and haloalkoxy.

The terms benzyl and phenylmethyl are interchangeable.

The term “heterocycloalkyl” embraces saturated and partially unsaturated heterocyclo-substituted alkyl radicals, such as pyrrolidinylmethyl, and heteroarylsubstituted alkyl radicals, such as pyridylmethyl, quinolylmethyl, thienylmethyl, furylethyl, and quinolylethyl. The heteroaryl in said heteroaralkyl may be additionally substituted with halo, alkyl, alkoxy, haloalkyl and haloalkoxy.

The term “aralkoxy” embraces aralkyl radicals attached through an oxygen atom to other radicals. The term “aralkoxyalkyl” embraces aralkoxy radicals attached through an oxygen atom to an alkyl radical. The term “aralkylthio” embraces aralkyl radicals attached to a sulfur atom. The term “aralkylthioalkyl” embraces aralkylthio radicals attached through a sulfur atom to an alkyl radical.

The term “aminoalkyl” embraces alkyl radicals substituted with one or more amino radicals. More preferred are “lower aminoalkyl” radicals. Examples of such radicals include aminomethyl, aminoethyl, and the like. The term “alkylamino” denotes amino groups that have been substituted with one or two alkyl radicals. Preferred are “lower N-alkylamino” radicals having alkyl portions having 1 to 6 carbon atoms. Suitable lower alkylamino may be mono or dialkylamino such as N-methylamino, N-ethylamino, N.N-dimethylamino, N,N-diethylamino or the like. The term “arylamino” denotes amino groups that have been substituted with one or two aryl radicals, such as N-phenylamino. The “arylamino” radicals may be further substituted on the aryl ring portion of the radical. The term “aralkylamino” embraces aralkyl radicals attached through an amino nitrogen atom to other radicals. The terms “N-arylaminoalkyl” and “N-aryl-N-alkylaminoalkyl” denote amino groups which have been substituted with one aryl radical or one aryl and one alkyl radical, respectively, and having the amino group attached to an alkyl radical. Examples of such radicals include N-phenylaminomethyl and N-phenyl-N-methylaminomethyl.

The term “aminocarbonyl” denotes an amide group of the formula —C(═O)NH₂. The term “alkylaminocarbonyl” denotes an aminocarbonyl group that has been substituted with one or two alkyl radicals on the amino nitrogen atom. Preferred are “N-alkylaminocarbonyl” and “N,N-dialkylaminocarbonyl” radicals. More preferred are “lower N-alkylaminocarbonyl” and “lower N,N-dialkylaminocarbonyl” radicals with lower alkyl portions as defined above. The term “aminocarbonylalkyl” denotes a carbonylalkyl group that has been substituted with an amino radical on the carbonyl carbon atom.

The term “alkylaminoalkyl” embraces radicals having one or more alkyl radicals attached to an aminoalkyl radical. The term “aryloxyalkyl” embraces radicals having an aryl radical attached to an alkyl radical through a divalent oxygen atom. The term “arylthioalkyl” embraces radicals having an aryl radical attached to an alkyl radical through a divalent sulfur atom.

Details

Among its several embodiments, the present invention provides a therapeutic method for treating or preventing an ocular COX-2 mediated disorder comprising administering a source of a COX-2 inhibitor compound to a mammal in need of such treatment, where the disorder is selected from blepharitis, post-operative inflammation and pain from corneal transplant surgery, endophthalmitis, episcleritis, keratitis, keratoconjunctivitis, keratoconjunctivitis sicca, post-operative inflammation and pain from lens implantation surgery, Mooren's ulcer, and post-operative inflammation and pain from retinal detachment surgery. Preferred COX-2 inhibitors are celecoxib, deracoxib, valdecoxib, a benzopyran COX-2 inhibitor, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methylbutoxy)-5-[4-(methylsulfonyl)phenyl]-3(2H)-pyridazinone.

In another embodiment, the source of the COX-2 inhibitor compound is a prodrug of a COX-2 inhibitor compound, illustrated herein with parecoxib.

In yet another embodiment, the invention provides a therapeutic method for treating or preventing an ocular COX-2 mediated disorder comprising administering celecoxib to a mammal in need of such treatment, where the disorder is selected from macular edema, intraoperative miosis, and ocular pain.

In still another embodiment, the invention provides a therapeutic method for treating or preventing an ocular COX-2 mediated disorder comprising administering deracoxib to a mammal in need of such treatment, where the disorder is selected from post-operative inflammation and pain from cataract surgery, acute injury to the eye tissue, glaucoma, macular edema, intraoperative miosis, ocular pain, photophobia, post-operative inflammation and pain from refractive surgery, retinitis, retinopathies and uveitis.

In another embodiment, the invention provides a therapeutic method for treating or preventing an ocular COX-2 mediated disorder comprising administering valdecoxib to a mammal in need of such treatment, where the disorder is selected from macular edema, intraoperative miosis and ocular pain.

In yet another embodiment, the invention provides a therapeutic method for treating or preventing an ocular COX-2 mediated disorder comprising administering a benzopyran COX-2 inhibitor to a mammal in need of such treatment, where the disorder is selected from glaucoma, macular edema, intraoperative miosis and ocular pain.

In still another embodiment, the invention provides a therapeutic method for treating or preventing an ocular COX-2 mediated disorder comprising administering parecoxib to a mammal in need of such treatment, where the disorder is selected from conjunctivitis, glaucoma, macular edema, intraoperative miosis and ocular pain.

In another embodiment, the invention provides a therapeutic method for treating or preventing an ocular COX-2 mediated disorder comprising administering rofecoxib to a mammal in need of such treatment, where the disorder is selected from post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, sarcoidosis and uveitis.

In yet another embodiment, the invention provides a therapeutic method for treating or preventing an ocular COX-2 mediated disorder comprising administering etoricoxib to a mammal in need of such treatment, where the disorder is selected from post-operative inflammation and pain from cataract surgery, conjunctivitis, acute injury to the eye tissue, macular edema, intraoperative miosis, ocular pain, photophobia, post-operative inflammation and pain from refractive surgery, retinitis, retinopathies, sarcoidosis and uveitis.

In still another embodiment, the invention provides a therapeutic method for treating or preventing an ocular COX-2 mediated disorder comprising administering 2-(3,5-difluorophenyl)-3-[4-(methylsulfonyl)phenyl]-2-cyclopenten-1-one to a mammal in need of such treatment, where the disorder is selected from post-operative inflammation and pain from cataract surgery, conjunctivitis, acute injury to the eye tissue, macular edema, intraoperative miosis, ocular pain, photophobia, post-operative inflammation and pain from refractive surgery, retinitis, sarcoidosis and uveitis.

In another embodiment, the invention provides a therapeutic method for treating or preventing an ocular COX-2 mediated disorder comprising administering 2-(3,4-difluorophenyl)-4-(3-hydroxy-3-methylbutoxy)-5-[4-(methylsulfonyl)phenyl]-3(2H)-pyridazinone to a mammal in need of such treatment, where the disorder is selected from post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, retinopathies, sarcoidosis and uveitis.

In yet another embodiment, the invention provides a pharmaceutical composition for treating or preventing Mooren's ulcer, in a mammal in need of such treatment, consisting essentially of a source of a COX-2 inhibitor compound and one or more ophthalmically acceptable excipient ingredients that reduce the rate of removal of the composition from the eye by lacrimation such that the composition has an effective residence time in the eye of about 2 to about 24 hours.

Types of retinopathies treated or prevented by the methods of the invention include, but are not limited to, hypertensive retinopathy and diabetic retinopathy. Types of macular edema treated or prevented by the methods of the invention include, but are not limited to, cystoid macular edema and macular edema associated with diabetic retinopathy. Ocular pain and ocular inflammation may be treated or prevented by the methods of the invention. Ocular pain and ocular inflammation treated or prevented by the methods of the invention may be related to acute or chronic injury to the eye tissue.

Besides being useful for human treatment, these methods are also useful for veterinary treatment of companion animals, exotic animals and farm animals, including mammals, rodents, avians, and the like. More preferred animals include horses, cows, dogs, cats, rats, mice, sheep and pigs.

The following references listed in Table No. 1 below, hereby individually incorporated by reference, describe various COX-2 inhibitors suitable for use in the present invention described herein, and processes for their manufacture.

TABLE NO 1
COX-2 Inhibitor References

| | | | |
|---|---|---|---|
| CA 21/80624 | DE 19/753463 | DE 38/34204 | EP 595546 |
| EP 921119 | FR 27/51966 | FR 27/70131 | FR 27/71005 |
| GB 22/83745 | GB 22/94879 | GB 23/19772 | GB 23/30833 |
| JP 09/052882 | JP 10/175861 | US 3840597 | US 4233299 |
| US 5733909 | US 5760068 | US 5824699 | US 5859257 |
| US 5968974 | WO 94/13635 | WO 94/20480 | WO 94/25431 |
| WO 94/27980 | WO 95/00501 | WO 95/11883 | WO 95/15315 |
| WO 95/15316 | WO 95/21817 | WO 95/30652 | WO 95/30656 |
| WO 96/03387 | WO 96/03388 | WO 96/03392 | WO 96/06840 |
| WO 96/09304 | WO 96/13483 | WO 96/16934 | WO 96/21667 |
| WO 96/23786 | WO 96/25405 | WO 96/25928 | WO 96/36617 |
| WO 96/37467 | WO 96/37469 | WO 96/41625 | WO 96/41626 |
| WO 96/41645 | WO 97/03667 | WO 97/03678 | WO 97/11701 |
| WO 97/27181 | WO 97/28120 | WO 97/28121 | WO 97/29774 |
| WO 97/29775 | WO 97/29776 | WO 97/36497 | WO 97/36863 |
| WO 97/37984 | WO 97/38986 | WO 97/40012 | WO 97/44027 |
| WO 97/44028 | WO 98/05639 | WO 98/06715 | WO 98/11080 |
| WO 98/15528 | WO 98/16227 | WO 98/22101 | WO 98/25896 |
| WO 98/29382 | WO 98/37235 | WO 98/41864 | WO 98/53814 |
| WO 98/53817 | WO 98/53818 | WO 98/57966 | WO 99/05104 |
| WO 99/09988 | WO 99/13799 | WO 99/15503 | WO 99/18960 |
| WO 99/30721 | WO 99/30729 | ZA 97/04806 | |

The selective COX-2 inhibitory drug can be any such drug known in the art, including without limitation compounds disclosed in the patents and publications listed below, each of which is individually incorporated herein by reference.

U.S. Pat. No. 5,344,991 to Reitz & Li.

U.S. Pat. No. 5,380,738 to Norman et al.

U.S. Pat. No. 5,393,790 to Reitz et al.

U.S. Pat. No. 5,401,765 to Lee.

U.S. Pat. No. 5,418,254 to Huang & Reitz.

U.S. Pat. No. 5,420,343 to Koszyk & Weier.

U.S. Pat. No. 5,434,178 to Talley & Rogier.

U.S. Pat. No. 5,436,265 to Black et al.

U.S. Pat. No. 5,466,823.

U.S. Pat. No. 5,474,995.

U.S. Pat. No. 5,475,018 to Lee & Bertenshaw.

U.S. Pat. No. 5,486,534 to Lee et al.

U.S. Pat. No. 5,510,368 to Lau et al.

U.S. Pat. No. 5,521,207.

U.S. Pat. No. 5,521,213 to Prasit et al.

U.S. Pat. No. 5,536,752 to Ducharme et al.

U.S. Pat. No. 5,543,297 to Cromlish et al.

U.S. Pat. No. 5,547,975 to Talley et al.

U.S. Pat. No. 5,550,142 to Ducharme et al.

U.S. Pat. No. 5,552,422 to Gauthier et al.

U.S. Pat. No. 5,585,504 to Desmond et al.

U.S. Pat. No. 5,593,992 to Adams et al.

U.S. Pat. No. 5,596,008 to Lee.

U.S. Pat. No. 5,604,253 to Lau et al.

U.S. Pat. No. 5,604,260 to Guay & Li.

U.S. Pat. No. 5,616,458 to Lipsky et al.

U.S. Pat. No. 5,616,601 to Khanna et al.

U.S. Pat. No. 5,620,999 to Weier et al.

U.S. Pat. No. 5,633,272.

U.S. Pat. No. 5,639,780 to Lau et al.

U.S. Pat. No. 5,643,933 to Talley et al.

U.S. Pat. No. 5,658,903 to Adams et al.

U.S. Pat. No. 5,668,161 to Talley et al.

U.S. Pat. No. 5,670,510 to Huang & Reitz.

U.S. Pat. No. 5,677,318 to Lau.

U.S. Pat. No. 5,681,842 to Dellaria & Gane.

U.S. Pat. No. 5,686,460 to Nicolaï et al.

U.S. Pat. No. 5,686,470 to Weier et al.

U.S. Pat. No. 5,696,143 to Talley et al.

U.S. Pat. No. 5,710,140 to Ducharme et al.

U.S. Pat. No. 5,716,955 to Adams et al.

U.S. Pat. No. 5,723,485 to Güngör & Teulon.

U.S. Pat. No. 5,739,166 to Reitz et al.

U.S. Pat. No. 5,741,798 to Lazer et al.

U.S. Pat. No. 5,756,499 to Adams et al.

U.S. Pat. No. 5,756,529 to Isakson & Talley.

U.S. Pat. No. 5,776,967 to Kreft et al.

U.S. Pat. No. 5,783,597 to Beers & Wachter.

U.S. Pat. No. 5,789,413 to Black et al.

U.S. Pat. No. 5,807,873 to Nicolaï & Teulon.

U.S. Pat. No. 5,817,700 to Dube et al.

U.S. Pat. No. 5,830,911 to Failli et al.

U.S. Pat. No. 5,849,943 to Atkinson & Wang.

U.S. Pat. No. 5,859,036 to Sartori et al.

U.S. Pat. No. 5,861,419.

U.S. Pat. No. 5,866,596 to Sartori & Teulon.

U.S. Pat. No. 5,869,524 to Failli.

U.S. Pat. No. 5,869,660 to Adams et al.

U.S. Pat. No. 5,883,267 to Rossen et al.

U.S. Pat. No. 5,892,053 to Zhi et al.

U.S. Pat. No. 5,916,905.

U.S. Pat. No. 5,922,742 to Black et al.

U.S. Pat. No. 5,929,076 to Adams & Garigipati.

U.S. Pat. No. 5,932,598.

U.S. Pat. No. 5,935,990 to Khanna et al.

U.S. Pat. No. 5,945,539 to Haruta et al.

U.S. Pat. No. 5,958,978 to Yamazaki et al.

U.S. Pat. No. 5,968,958 to Guay et al.

U.S. Pat. No. 5,972,950 to Nicolaï & Teulon.

U.S. Pat. No. 5,973,191 to Marnett & Kalgutkar.

U.S. Pat. No. 5,981,576.

U.S. Pat. No. 5,994,381 to Haruta et al.

U.S. Pat. No. 6,002,014 to Haruta et al.

U.S. Pat. No. 6,004,960 to Li et al.

U.S. Pat. No. 6,005,000 to Hopper et al.

U.S. Pat. No. 6,020,343 to Belley et al.

U.S. Pat. No. 6,020,347 to DeLaszlo & Hagmann.

U.S. Pat. No. 6,034,256.

U.S. Pat. No. 6,040,319 to Corley et al.

U.S. Pat. No. 6,040,450 to Davies et al.

U.S. Pat. No. 6,046,208 to Adams et al.

U.S. Pat. No. 6,046,217 to Friesen et al.

U.S. Pat. No. 6,057,319 to Black et al.

U.S. Pat. No. 6,063,804 to De Nanteuil et al.

U.S. Pat. No. 6,063,807 to Chabrier de Lassauniere & Broquet.

U.S. Pat. No. 6,071,954 to LeBlanc et al.

U.S. Pat. No. 6,077,868 to Cook et al.

U.S. Pat. No. 6,077,869 to Sui & Wachter.

U.S. Pat. No. 6,083,969 to Ferro et al.

U.S. Pat. No. 6,096,753 to Spohr et al.

U.S. Pat. No. 6,133,292 to Wang et al.

International Patent Publication No. W: 94/15932.

International Patent Publication No. WO 96/03385.

International Patent Publication No. WO 96/19469.

International Patent Publication No. WO 96/24584.

International Patent Publication No. WO 96/24585.

International Patent Publication No. WO 96/26921.

International Patent Publication No. W. 96/31509.

International Patent Publication No. WO 96/36623.

International Patent Publication No. WO 96/38418.

International Patent Publication No. WO 96/38442.

International Patent Publication No. WO 97/03953.

International Patent Publication No. WO 97/10840.

International Patent Publication No. WO 97/13755.

International Patent Publication No. WO 97/13767.

International Patent Publication No. WO 97/14691.

International Patent Publication No. WO 97/16435.

International Patent Publication No. WO 97/25048.

International Patent Publication No. WO 97/30030.

International Patent Publication No. WO 97/34882.

International Patent Publication No. WO 97/46524.

International Patent Publication No. WO 98/03484.

International Patent Publication No. WO 98/04527.

International Patent Publication No. WO 98/06708.

International Patent Publication No. WO 98/07425.

International Patent Publication No. WO 98/17292.

International Patent Publication No. WO 98/21195.

International Patent Publication No. WO 98/22457.

International Patent Publication No. WO 98/32732.

International Patent Publication No. Wo 98/41511.

International Patent Publication No. WO 98/41516.

International Patent Publication No. WO 98/43966.

International Patent Publication No. WO 98/45294.

International Patent Publication No. WO 98/47871.

International Patent Publication No. WO 98/47890.

International Patent Publication No. Wo 99/01130.

International Patent Publication No. WO 99/01131.

International Patent Publication No. WO 99/01452.

International Patent Publication No. Wo 99/01455.

International Patent Publication No. Wo 99/10331.

International Patent Publication No. WO 99/10332.

International Patent Publication No. Wo 99/11605.

International Patent Publication No. Wo 99/12930.

International Patent Publication No. Wo 99/14194.

International Patent Publication No. Wo 99/14195.

International Patent Publication No. Wo 99/14205.

International Patent Publication No. Wo 99/15505.

International Patent Publication No. Wo 99/15513.

International Patent Publication No. Wo 99/23087.

International Patent Publication No. Wo 99/24404.

International Patent Publication No. WO 99/25695.

International Patent Publication No. Wo 99/35130.

International Patent Publication No. WO 99/61016.

International Patent Publication No. WO 99/61436.

International Patent Publication No. WO 99/62884.

International Patent Publication No. WO 99/64415.

International Patent Publication No. WO 00/01380.

International Patent Publication No. WO 00/08024.

International Patent Publication No. WO 00/10993.

International Patent Publication No. WO 00/13684.

International Patent Publication No. WO 00/18741.

International Patent Publication No. WO 00/18753.

International Patent Publication No. W, 00/23426.

International Patent Publication No. WO 00/23433.

International Patent Publication No. W. 00/24719.

International Patent Publication No. WO 00/26216.

International Patent Publication No. WO 00/31072.

International Patent Publication No. WO 00/32189.

International Patent Publication No. We 00/40087.

International Patent Publication No. WO 00/56348.

International Patent Publication No. WO 00/66562.

European Patent Application No. 0 799 823.

Europea n Patent Application No. 0 846 689.

Europea n Patent Application No. 0 863 134.

European Patent Application No. 0 924 201.

European Patent Application No. 0 985 666.

European Patent Application No. 0 995 747.

Three classes of COX-2 inhibitors are reviewed by J. Carter in Exp. Opin. Ther. Patents, 8(1), 21-29 (1997): methanesulfonanilides, tricyclics and structurally modified non-selective cyclooxygenase inhibitors. Methanesulfonanilides are a class of selective COX-2 inhibitors, of which NS-398, flosulide and nimesulide are example members.

A preferred class of tricyclic COX-2 inhibitors comprises compounds of formula (VII)

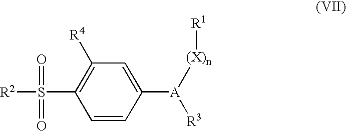

wherein A is a substituent selected from partially unsaturated or unsaturated heterocyclyl and partially unsaturated or unsaturated carbocyclic rings;

wherein n is 0 or 1;

wherein X is O, S or CH₂;

wherein R¹ is at least one substituent selected from heterocyclyl, cycloalkyl, cycloalkenyl and aryl, wherein R¹ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

wherein R² is methyl, amino or aminocarbonylalkyl; and

wherein R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, wherein R³ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

wherein R⁴ is selected from hydrido and halo;

or a pharmaceutically-acceptable salt thereof.

More preferred COX-2 inhibitors are tricyclic COX-2 inhibitors wherein the A ring of formula (VII) is selected from the heterocyclyl groups of pyrazolyl, furanonyl, isoxazolyl, pyridinyl, cyclopentenonyl and pyridazinonyl.

Further preferred COX-2 inhibitors that may be used in the present invention include, but are not limited to:

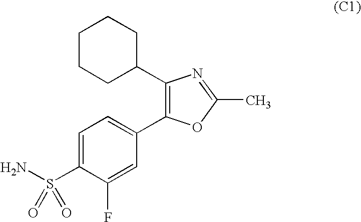

JTE-522, 4-(4-cyclohexyl-2-methyloxazol-5-yl)-2-fluorobenzenesulfonamide;

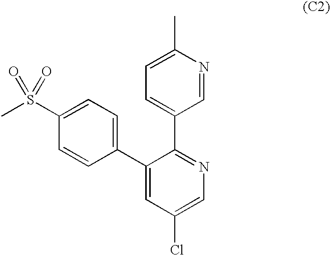

5-chloro-3-(4-(rnethylsulfonyl)phenyl)-2-(2-methyl-5-pyridinyl) pyridine;

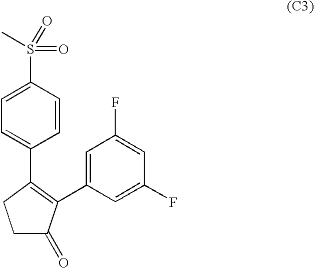

2-(3,5-difluorophenyl)-3-(4-(methylsulfonyl)phenyl)-2-cyclopenten-1-one;

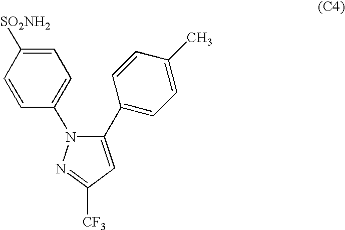

celecoxib, 4-[5-(4-methylphenyl)-3-(trifluoromethyl)-1H-pyrazol-1-yl]-benzenesulfonamide;

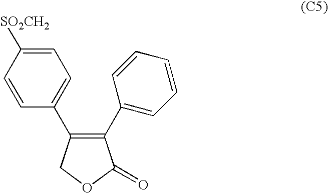

rofecoxib, 4-[4-(methylsulfonyl)phenyl]-3-phenyl-2 (5H)-furanone;

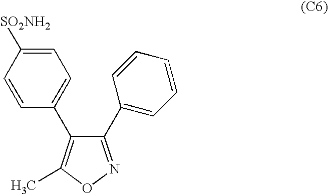

valdecoxib, 4-(5-methyl-3-phenylisoxazol-4-yl)benzenesulfonamide;

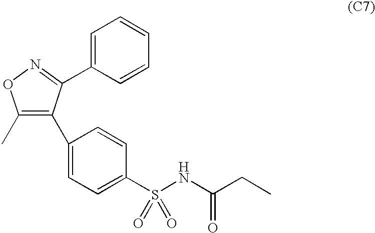

parecoxib, N-[[4-[5-methyl-3-phenylisoxazol-4-yl]phenyl]sulfonyl]propanamide;

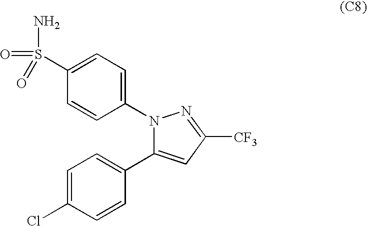

4-[5-(4-chorophenyl)-3-(trifluoromethyl)-1H-pyrazole-1-yl]benzenesulfonamide;

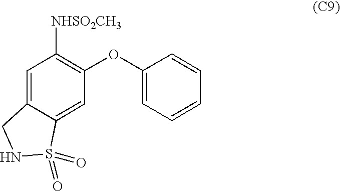

N-(2,3-dihydro-1,1-dioxido-6-phenoxy-1,2-benzisothiazol-5-yl)methanesulfonamide;

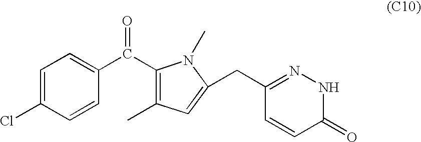

6-[[5-(4-chlorobenzoyl)-1,4-dimethyl-1H-pyrrol-2-yl]methyl]-3(2H)-pyridazinone;

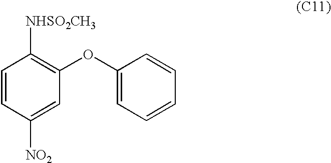

N-(4-nitro-2-phenoxyphenyl)methanesulfonamide;

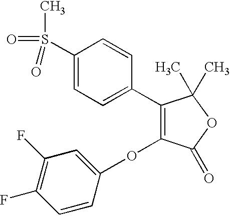

3-(3,4-difluorophenoxy)-5,5-dimethyl-4-[4-(methylsulfonyl)phenyl]-2(5H)-furanone;

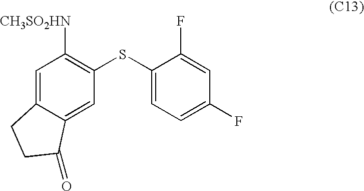

N-[6-[(2,4-difluorophenyl)thio]-2,3-dihydro-1-oxo-1H-inden-5-yl]methanesulfonamide;

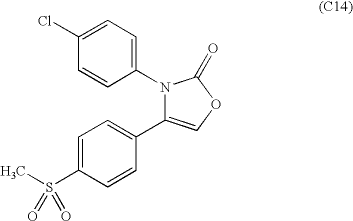

3-(4-chlorophenyl)-4-[4-(methylsulfonyl)phenyl]-2(3H)-oxazolone;

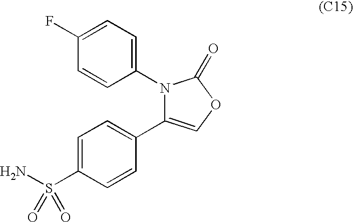

4-[3-(4-fluorophenyl)-2,3-dihydro-2-oxo-4-oxazolyl]benzenesulfonamide;

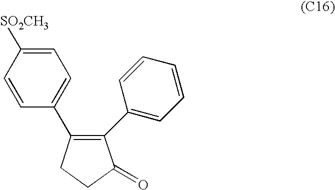

3-[4-(methylsulfonyl)phenyl]-2-phenyl-2-cyclopenten-1-one;

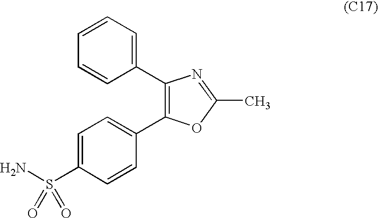

4-(2-methyl-4-phenyl-5-oxazolyl)benzenesulfonamide;

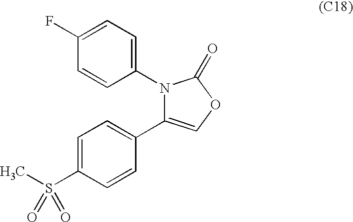

3-(4-fluorophenyl)-4-[4-(methylsulfonyl)phenyl]-2(3H)-oxazolone;

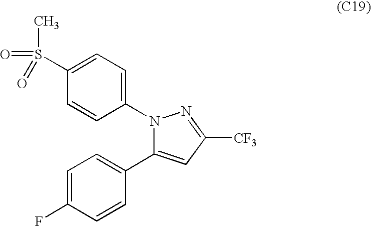

5-(4-fluorophenyl)-1-[4-(methylsulfonyl)phenyl]-3-(trifluoromethyl)-1H-pyrazole;

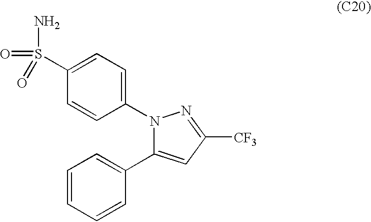

4-[5-phenyl)-3-(trifluoromethyl)-1H-pyrazol-1-yl)benzenesulfonamide;

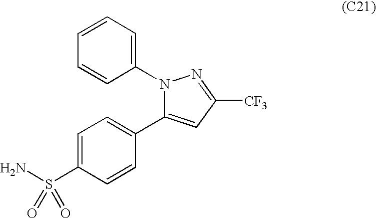

4-[1-phenyl-3-(trifluoromethyl)-1H-pyrazol-5-yl]benzenesulfonamide;

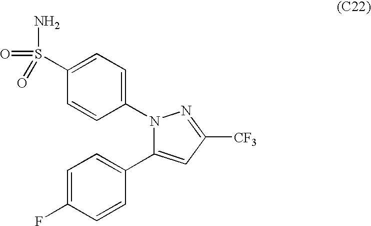

4-[5-(4-fluorophenyl)-3-(trifluoromethyl)-1H-pyrazol-1-yl]benzenesulfonamide;

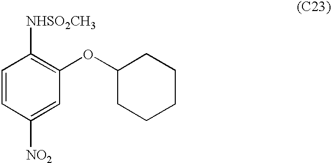

NS-398, N-[2-(cyclohexyloxy)-4-nitrophenyl]methanesulfonamide;

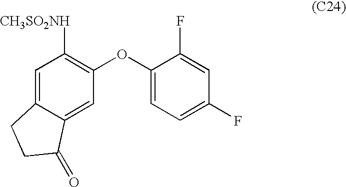

N-[6-(2,4-difluorophenoxy)-2,3-dihydro-1-oxo-1H-inden-5-yl]methanesulfonamide;

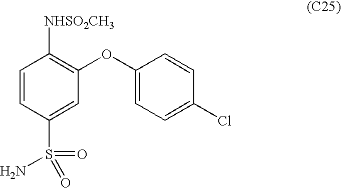

3-(4-chlorophenoxy)-4-[(methylsulfonyl)amino]benzenesulfonamide;

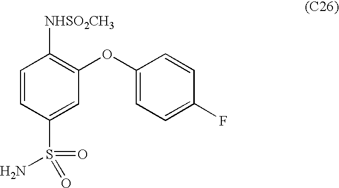

3-(4-fluorophenoxy)-4-[(methylsulfonyl)amino]benzenesulfonamide;

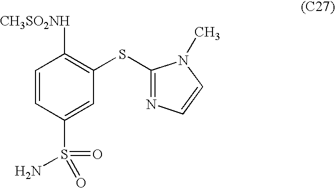

3-[(1-methyl-1H-imidazol-2-yl)thio]-4[(methylsulfonyl) amino]benzenesulfonamide;

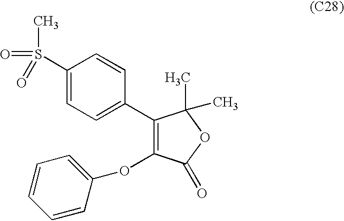

5,5-dimethyl-4-[4-(methylsulfonyl)phenyl]-3-phenoxy-2 (5H)-furanone;

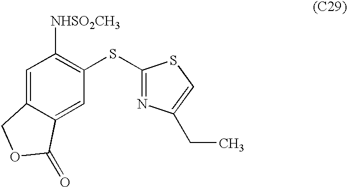

N-[6-[(4-ethyl-2-thiazolyl)thio]-1,3-dihydro-1-oxo-5-isobenzofuranyl]methanesulfonamide;

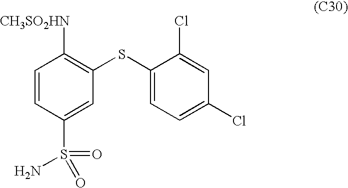

3-[(2,4-dichlorophenyl)thio]-4-[(methylsulfonyl)amino]benzenesulfonamide;

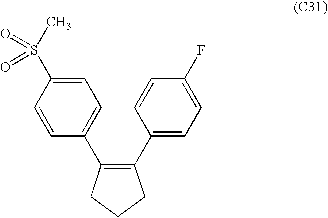

1-fluoro-4-[2-[4-(methylsulfonyl)phenyl]cyclopenten-1-yl]benzene;

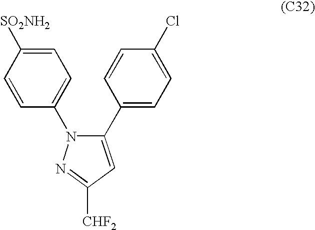

4-[5-(4-chlorophenyl)-3-(difluoromethyl)-1H-pyrazol-1-yl]benzenesulfonamide;

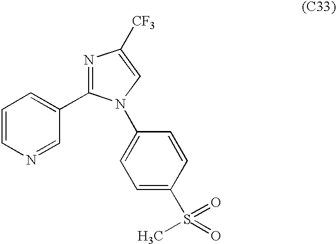

3-[1-[4-(methylsulfonyl)phenyl]-4-(trifluoromethyl)-1H-imidazol-2-yl]pyridine;

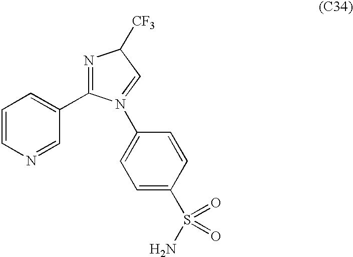

4-[2-(3-pyridinyll)-4-(trifluoromethyl)-1H-imidazol-1-yl]benzenesulfonamide;

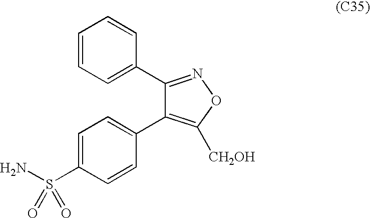

4-[5-(hydroxymethyl)-3-phenylisoxazol-4-yl]benzenesulfonamide;

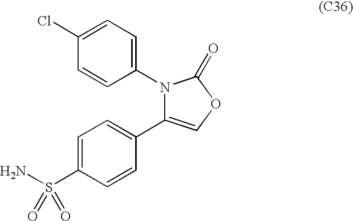

4-[3-(4-chlorophenyl)-2,3-dihydro-2-oxo-4-oxazolyl]benzenesulfonamide;

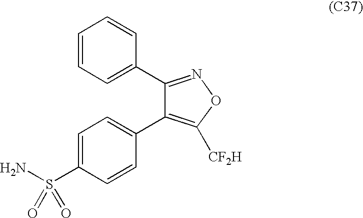

4-[5-(difluoromethyl)-3-phenylisoxazol-4-yl]benzenesulfonamide;

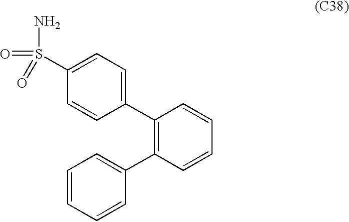

[1,1′:2′,1″-terphenyl]-4-sulfonamide;

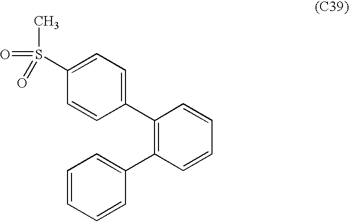

4-(methylsulfonyl)-1,1′,2],1″-terphenyl;

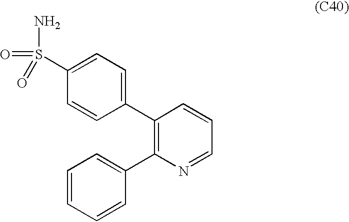

4-(2-phenyl-3-pyridinyl) benzenesulfonamide;

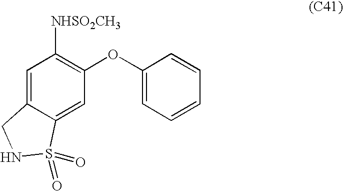

N-(2,3-dihydro-1,1-dioxido-6-phenoxy-1,2-benzisothiazol-5-yl)methanesulfonamide;

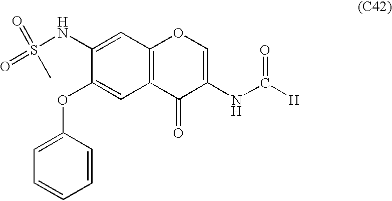

N-[3-(formylamino)-4-oxo-6-phenoxy-4H-1-benzopyran-7-yl]methanesulfonamide;

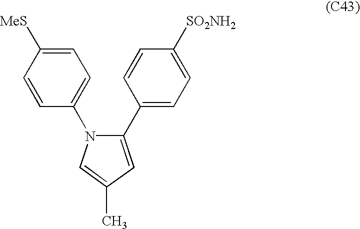

4-[4-methyl-1-[4-(methylthio)phenyl]-1H-pyrrol-2-yl]benzenesulfonamide;

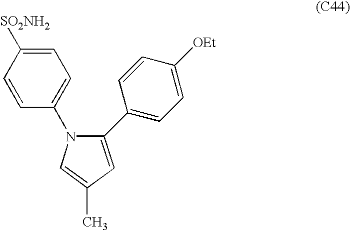

4-[2-(4-ethoxyphenyl)-4-methyl-1H-pyrrol-1-yl]benzenesulfonamide;

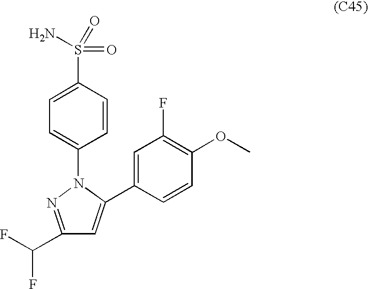

deracoxib, 4-[3-(difluoromethyl)-5-(3-fluoro-4-methoxyphenyl)-1H-pyrazol-1-yl]benzenesulfonamide;

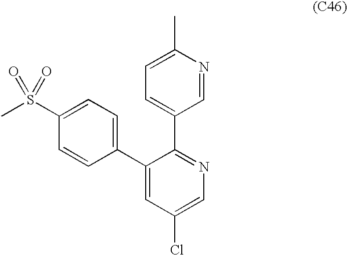

MK-663, etoricoxib, 5-chloro-6′-methyl-3-[4-(methylsulfonyl)phenyl]-2,3′-bipyridine;

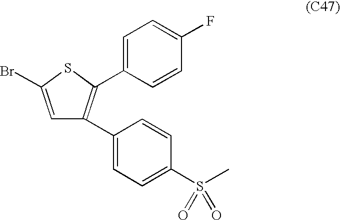

DuP 697, 5-bromo-2-(4-fluorop henyl)-3-[4-(methylsulfonyl)phenyl]thiophene;

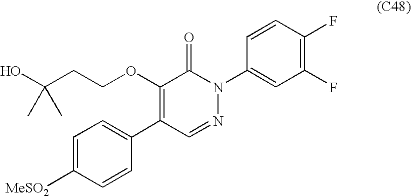

ABT-963, 2-(3,4-difluorophenyl)-4-(3-hydroxy-3-methylbutoxy)-5-[4-(methylsulfonyl)phenyl]-3 (2H)-pyridazinone;

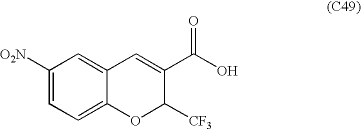

6-nitro-2-trifluoromethyl-2H-1-benzopyran-3-carboxylic acid;

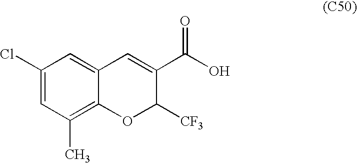

6-chloro-8-methyl-2-trifluoromethyl-2H-1-benzopyran-3-carboxylic acid;

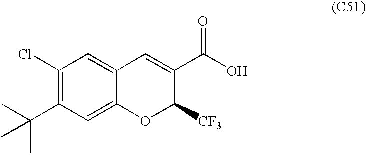

(2S)-6-chloro-7-(1,1-dimethylethyl)-2-(trifluoromethyl)-2H-1-benzopyran-3-carboxylic acid;

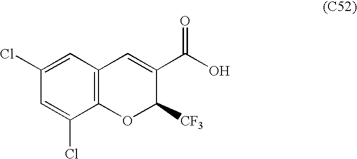

(2S)-6,8-dichloro-2-(trifluoromethyl)-2H-1-benzopyran-3-carboxylic acid;

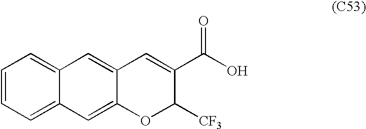

2-trifluoromethyl-2H-naphtho[2,3-b]pyran-3-carboxylic acid;

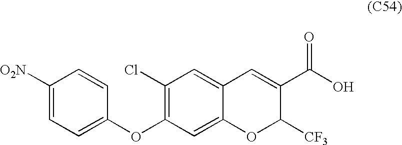

6-chloro-7-(4-nitrophenoxy)-2-(trifluoromethyl)-2H-1-benzopyran-3-carboxylic acid;

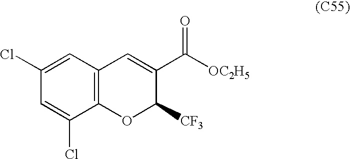

(2S)-6,8-dichloro-2-(trifluoromethyl)-2H-1-benzopyran-3-carboxylic acid, ethyl ester;

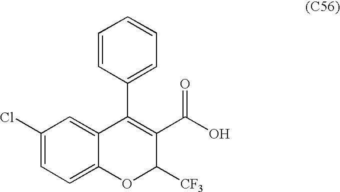

6-chloro-2-(trifluoromethyl)-4-phenyl-2H-1-benzopyran-3-carboxylic acid;

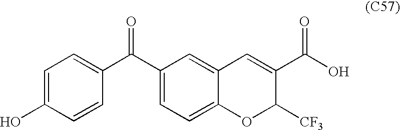

6-(4-hydroxybenzoyl)-2-(trifluoromethyl)-2H-1-benzopyran-3-carboxylic acid;

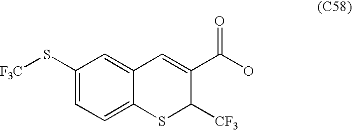

2-(trifluoromethyl)-6-[(trifluoromethyl)thio]-2H-1-benzothiopyran-3-carboxylic acid;

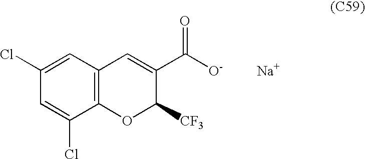

(2S)-6,8-dichloro-2-(trifluoromethyl)-2H-1-benzopyran-3-carboxylic acid, sodium salt;

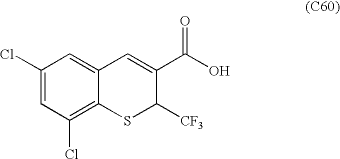

6,8-dichloro-2-trifluoromethyl-2H-1-benzothiopyran-3-carboxylic acid;

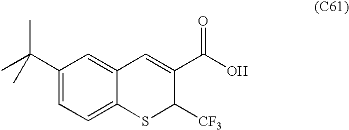

6-(1,1-dimethylethyl)-2-(trifluoromethyl)-2H-1-benzothiopyran-3-carboxylic acid;

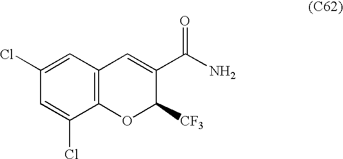

(2S)-6,8-dichloro-2-(trifluoromethyl)-2H-1-benzopyran-3-carboxamide;

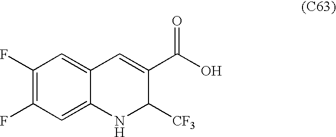

6,7-difluoro-1,2-dihydro-2-(triflucromethyl)-3-quinolinecarboxylic acid;

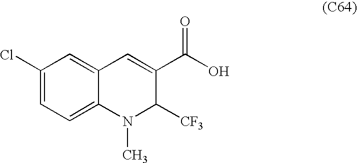

6-chloro-1,2-dihydro-1-methyl-2-(trifluoromethyl)-3-quinolinecarboxylic acid;

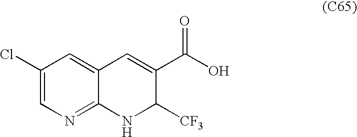

6-chloro-2-(trifluoromethyl)-1,2-dihydro[1,8]naphthyridine-3-carboxylic acid;

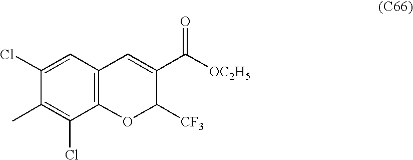

6,8-dichloro-7-methyl-2-(trifluoromethyl)-2H-1-benzopyran-3-carboxylic acid, ethyl ester;

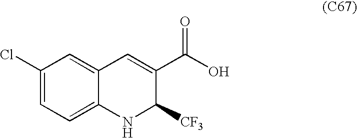

(2S)-6-chloro-1,2-dihydro-2-(trifluoromethyl)-3-quinolinecarboxylic acid;

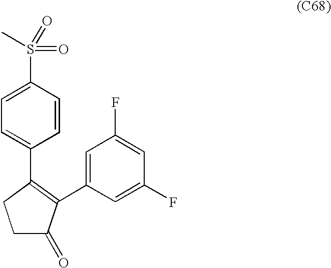

L-776,967, 2-(3,5-difluorophenyl)-3-[4-(methylsulfonyl)phenyl]-2-cyclopenten-1-one.

The CAS reference numbers for nonlimiting examples of COX-2 inhibitors are identified in Table 2 below.

TABLE NO 2
COX-2 Inhibitors

| Compound Number | CAS Reference Number |
|---|---|
| C1 | 180200-68-4 |
| C2 | 202409-33-4 |
| C3 | 212126-32-4 |
| C4 | 169590-42-5 |
| C5 | 162011-90-7 |
| C6 | 181695-72-7 |
| C7 | 198470-84-7 |
| C8 | 170569-86-5 |
| C9 | 187845-71-2 |
| C10 | 179382-91-3 |
| C11 | 51803-78-2 |
| C12 | 189954-13-0 |
| C13 | 158205-05-1 |
| C14 | 197239-99-9 |
| C15 | 197240-09-8 |
| C16 | 226703-01-1 |
| C17 | 93014-16-5 |
| C18 | 197239-97-7 |
| C19 | 162054-19-5 |
| C20 | 170569-87-6 |
| C21 | 279221-13-5 |
| C22 | 170572-13-1 |
| C23 | 123653-11-2 |
| C24 | 80937-31-1 |
| C25 | 279221-14-6 |
| C26 | 279221-15-7 |
| C27 | 187846-16-8 |
| C28 | 189954-16-3 |
| C29 | 181485-41-6 |
| C30 | 187845-80-3 |
| C31 | 158959-32-1 |
| C32 | 170570-29-3 |
| C33 | 177660-77-4 |
| C34 | 177660-95-6 |
| C35 | 181695-81-8 |
| C36 | 197240-14-5 |
| C37 | 181696-33-3 |
| C38 | 178816-94-9 |
| C39 | 178816-61-0 |
| C40 | 279221-17-9 |
| C41 | 187845-71-2 |
| C42 | 123663-49-0 |
| C43 | 197905-01-4 |
| C44 | 197904-84-0 |
| C45 | 169590-41-4 |
| C46 | 202409-33-4 |
| C47 | 88149-94-4 |
| C48 | 266320-83-6 |
| C49 | 215122-43-3 |
| C50 | 215122-44-4 |
| C51 | 215122-74-0 |
| C52 | 215123-80-1 |
| C53 | 215122-70-6 |
| C54 | 264878-87-7 |
| C55 | 279221-12-4 |
| C56 | 215123-48-1 |
| C57 | 215123-03-8 |
| C58 | 215123-60-7 |
| C59 | 279221-18-0 |
| C60 | 215123-61-8 |
| C61 | 215123-52-7 |
| C62 | 279221-19-1 |
| C63 | 215123-64-1 |
| C64 | 215123-70-9 |
| C65 | 215123-79-8 |
| C66 | 215123-91-4 |
| C67 | 215123-77-6 |
| C68 | 212126-32-4 |

More preferably, the COX-2 inhibitors that may be used in the present invention include, but are not limited to celecoxib, deracoxib, valdecoxib, benzopyran COX-2 inhibitors, parecoxib, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methylbutoxy)-5-[4-(methylsulfonyl)phenyl]-3(2H)-pyridazinone.

Parecoxib can be used in the present invention in the form of a salt, for example, sodium parecoxib.

Various classes of COX-2 inhibitors can be prepared as follows. Pyrazoles can be prepared by methods described in WO 95/15316. Pyrazoles can further be prepared by methods described in WO 95/15315. Pyrazoles can also be prepared by methods described in WO 96/03385.

Thiophene analogs can be prepared by methods described in WO 95/00501. Preparation of thiophene analogs is also described in WO 94/15932.

Oxazoles can be prepared by the methods described in WO 95/00501. Preparation of oxazoles is also described in WO 94/27980.

Isoxazoles can be prepared by the methods described in WO 96/25405.

Imidazoles can be prepared by the methods described in WO 96/03388. Preparation of imidazoles is also described in WO 96/03387.

Cyclopentene COX-2 inhibitors can be prepared by the methods described in U.S. Pat. No. 5,344,991. Preparation of cyclopentene COX-2 inhibitors is also described in WO 95/00501.

Terphenyl compounds can be prepared by the methods described in WO 96/16934.

Thiazole compounds can be prepared by the methods described in WO 96/03,392.

Pyridine compounds can be prepared by the methods described in WO 96/03392. Preparation of pyridine compounds is also described in WO 96/24,585.

Benzopyranopyrazolyl compounds can be prepared by the methods described in WO 96/09304.

Benzopyran compounds can be prepared by the methods described in WO 98/47890. Preparation of benzopyran compounds is also described in WO 00/23433. Benzopyran compounds can further be prepared by the methods described in U.S. Pat. No. 6,077,850. Preparation of benzopyran compounds is further described in U.S. Pat. No. 6,034,256.

Arylpyridazinones can be prepared by the methods described in WO 00/24719. Preparation of arylpyridazinones is also described in WO 99/10332. Arylpyridazinones can further be prepared by the methods described in WO 99/10331.

The celecoxib used in the therapeutic methods of the present invention can be prepared in the manner set forth in U.S. Pat. No. 5,466,823.

The valdecoxib used in the therapeutic methods of the present invention can be prepared in the manner set forth in U.S. Pat. No. 5,633,272.

The parecoxib used in the therapeutic methods of the present invention can be prepared in the manner set forth in U.S. Pat. No. 5,932,598.

The rofecoxib used in the therapeutic methods of the present invention can be prepared in the manner set forth in U.S. Pat. No. 5,474,995.

The deracoxib used in the therapeutic methods of the present invention can be prepared in the manner set forth in U.S. Pat. No. 5,521,207.

The etoricoxib used in the therapeutic methods of the present invention can be prepared in the manner set forth in WO 98/03484.

The compound 2-(3,4-difluorophenyl)-4-(3-hydroxy-3-methylbutoxy)-5-[4-(methylsulfonyl)phenyl]-3(2H)-pyridazinone used in the therapeutic methods of the present invention can be prepared in the manner set forth in WO 00/24719.

The compound 2-(3,5-difluorophenyl)-3-[4-(methylsulfonyl)phenyl]-2-cyclopenten-1-one used in the therapeutic methods of the present invention can be prepared in the manner set forth in EP 863134.

The compounds useful in the present invention can have no asymmetric carbon atoms, or, alternatively, the useful compounds can have one or more asymmetric carbon atoms. When the useful compounds have one or more asymmetric carbon atoms, they therefore include racemates and stereoisomers, such as diastereomers and enantiomers, in both pure form and in admixture. Such stereoisomers can be prepared using conventional techniques, either by reacting enantiomeric starting materials, or by separating isomers of compounds of the present invention.

Isomers may include geometric isomers, for example cis-isomers or trans-isomers across a double bond. All such isomers are contemplated among the compounds useful in the present invention.

The compounds useful in the present invention also include tautomers.

The compounds useful in the present invention also include their salts, solvates and prodrugs.

Dosages, Formulations and Routes of Administration

For the prophylaxis or treatment of the conditions referred to above, the compounds useful in the combinations and methods of the present invention can be used as the compound per se. Pharmaceutically acceptable salts are particularly suitable for medical applications because of their greater aqueous solubility relative to the parent compound. Such salts must clearly have a pharmaceutically acceptable anion or cation. Suitable pharmaceutically acceptable acid addition salts of the compounds of the present invention when possible include those derived from inorganic acids, such as hydrochloric, hydrobromic, phosphoric, metaphosphoric, nitric, sulfonic, and sulfuric acids, and organic acids such as formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, β-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The compounds useful in the present invention can be presented with an acceptable carrier in the form of a pharmaceutical composition. The carrier must, of course, be acceptable in the sense of being compatible with the other ingredients of the composition and must not be deleterious to the recipient. The carrier can be a solid or a liquid, or both, and is preferably formulated with the compound as a unit-dose composition, for example, a tablet, which can contain from 0.05% to 95% by weight of the active compound. Other pharmacologically active substances can also be present, including other compounds of the present invention. The pharmaceutical compositions of the invention can be prepared by any of the well-known techniques of pharmacy, consisting essentially of admixing the components.

Optionally, the combination of the present invention can comprise a composition comprising a COX-2 inhibiting compound and another therapeutic agent. In such a composition, the COX-2 inhibiting compound and the therapeutic agent can be present in a single dosage form, for example a pill, a capsule, or a liquid that contains both of the compounds.

These compounds can be administered by any conventional means available for use in conjunction with pharmaceuticals, either as individual therapeutic compounds or as a combination of therapeutic compounds.

The amount of compound which is required to achieve the desired biological effect will, of course, depend on a number of factors such as the specific compound chosen, the use for which it is intended, the mode of administration, and the clinical condition of the recipient.

Dosages

Dosage levels of COX-2 inhibitors on the order of about 0.1 mg to about 10,000 mg of the active ingredient compound are useful in the treatment of the above conditions, with preferred levels of about 1.0 mg to about 1,000 mg and even more preferred levels of about 5 mg to about 500 mg. The amount of active ingredient will vary depending upon the host treated and the particular mode of administration.

It is understood, however, that a specific dose level for any particular patient will depend upon a variety of factors including the activity of the specific compound employed, the age, body weight, general health, sex, diet, time of administration, rate of excretion, drug combination, and the severity of the particular disease being treated and form of administration.

Treatment dosages generally may be titrated to optimize safety and efficacy. Typically, dosage-effect relationships from in vitro initially can provide useful guidance on the proper doses for patient administration. Studies in animal models also generally may be used for guidance regarding effective dosages for treatment of cancers in accordance with the present invention. In terms of treatment protocols, it should be appreciated that the dosage to be administered will depend on several factors, including the particular agent that is administered, the route administered, the condition of the particular patient, etc. Generally speaking, one will desire to administer an amount of the compound that is effective to achieve a serum level commensurate with the concentrations found to be effective in vitro. Thus, where a compound is found to demonstrate in vitro activity at, e.g., 10 AM, one will desire to administer an amount of the drug that is effective to provide about a 10 μM concentration in vivo. Determination of these parameters is well within the skill of the art. These considerations, as well as effective formulations and administration procedures are well known in the art and are described in standard textbooks.

Formulations and Routes of Administration

The compounds of the present invention can be formulated as a pharmaceutical composition. Such a composition can then be administered orally, parenterally, by inhalation spray, rectally, or topically in dosage unit formulations containing conventional nontoxic pharmaceutically acceptable carriers, adjuvants, and vehicles as desired. Formulation of drugs is discussed in, for example, Hoover, John E., Remington's Pharmaceutical Sciences, Mack Publishing Co., Easton, Pennsylvania 1975. Another discussion of drug formulations can be found in Liberman, H. A. and Lachman, L., Eds., Pharmaceutical Dosage Forms, Marcel Decker, New York, N.Y., 1980.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, a contemplated inhibitor compound can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

The term parenteral as used herein includes subcutaneous injections, intravenous, intramuscular, intrasternal injection, or infusion techniques. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents mentioned for use in the formulations for oral administration. A contemplated therapeutic compound can be dissolved in water, polyethylene glycol, propylene glycol, ethanol, corn oil, cottonseed oil, peanut oil, sesame oil, benzyl alcohol, sodium chloride solution, or various buffers. Other adjuvants and modes of administration are well and widely known in the pharmaceutical art.

Suppositories for rectal administration of the drug can be prepared by mixing the drug with a suitable nonirritating excipient such as cocoa butter, synthetic mono-, di- or triglycerides, fatty acids and polyethylene glycols that are solid at ordinary temperatures but liquid at the rectal temperature and will therefore melt in the rectum and release the drug.

The ocular COX-2 mediated disorders may be treated by administering the desired COX-2 inhibitor directly to the eye by use of a pharmaceutical formulation consisting of a solution, cream, ointment, emulsion, suspension and slow release formulations.

Preparation of the composition can be carried out by mixing the active ingredients with an ophthalmologically compatible carrier. Such carrier compounds are known per se and there are a number of systems based on physiologic saline, oil solutions or ointments suggested in the literature for application of medicaments to the eye. The carrier or vehicle may furthermore contain ophthalmologically compatible preservatives such as e.g. benzalkonium chloride, surfactants, such as polysorbate 80, liposomes or polymers, for example, methyl cellulose, polyvinyl alcohol, polyvinyl pyrrolidone and hyaluronic acid. The latter substances may be used for increasing the viscosity of the solution. Furthermore, it is also possible to use soluble or insoluble drug inserts, for instance gels or gel type materials, in order to obtain a slow-release system.

Preferably the composition has an effective residence time in the eye of about 2 to about 24 hours, more preferably about 4 to about 24 hours and most preferably about 6 to about 24 hours.

Lacrimation is the production of tear fluid, and can remove matter from the eyes both by external wash-out and by lacrimal drainage into the nasopharyngeal cavity via the nasolacrimal ducts. By “effective residence time” herein is meant a period of time following application of the composition to the eye during which a substantial portion of the applied composition remains in situ and during which the drug is released therefrom in a therapeutically or prophylactically effective amount to tissues of the eye or to fluids secreted thereby.

The composition therefore provides sustained release over a period of at least about 2 hours. Optionally a portion of the selective COX-2 inhibitory drug can be present in the composition in immediate-release form so that the composition provides a combination of immediate and sustained release (herein referred to as “dual release”) of the drug.

It will be understood that certain COX-2 mediated disorders of the eye are disorders of surface tissues such as the conjunctiva, and that topical application of a selective COX-2 inhibitory drug to the eye therefore delivers the drug directly to its site of action in the case of such disorders. Other COX-2 mediated disorders of the eye are disorders of internal tissues such as the retina, in which case the drug has to move from the locus of administration to the targeted tissue. Administration of a composition of the invention to the eye generally results in direct contact of the drug with the cornea, through which at least a portion of the administered drug passes. The term “topical” as applied herein to ocular administration of a composition of the invention will be understood to embrace administration followed by corneal absorption as well as administration directly to a targeted surface tissue of the eye.

A composition of the invention can illustratively take the form of a liquid wherein the drug is present in solution, in suspension or both. The term “solution/suspension” herein refers to a liquid composition wherein a first portion of the drug is present in solution and a second portion of the drug is present in particulate form, in suspension in a liquid matrix. A liquid composition herein includes a gel. Preferably the liquid composition is aqueous. Alternatively, the composition can take the form of an ointment.

As a further alternative, the composition can take the form of a solid article that can be inserted between the eye and eyelid or in the conjunctival sac, where it releases the drug as described, for example, in U.S. Pat. Nos. 3,863,633 and 3,868,445, both to Ryde & Ekstedt, incorporated herein by reference. Release is to the lacrimal fluid that bathes the surface of the cornea, or directly to the cornea itself, with which the solid article is generally in intimate contact. Solid articles suitable for implantation in the eye in such fashion are generally composed primarily of polymers and can be biodegradable or non-biodegradable. Biodegradable polymers that can be used in preparation of ocular implants carrying a selective COX-2 inhibitory drug in accordance with the present invention include without restriction aliphatic polyesters such as polymers and copolymers of poly(glycolide), poly(lactide), poly(s-caprolactone), poly(hydroxybutyrate) and poly(hydroxyvalerate), polyamino acids, polyorthoesters, polyanhydrides, aliphatic polycarbonates and polyether lactones. Illustrative of suitable non-biodegradable polymers are silicone elastomers.

In a preferred embodiment, the composition is an aqueous solution, suspension or solution/suspension, which can be presented in the form of eye drops. By means of a suitable dispenser, a desired dosage of the drug can be metered by administration of a known number of drops into the eye. For example, for a drop volume of 25 μl, administration of 1-6 drops will deliver 25-150 μl of the composition. Aqueous compositions of the invention preferably contain from about 0.01% to about 50%, more preferably about 0.1% to about 20%, still more preferably about 0.2% to about 10%, and most preferably about 0.5% to about 5%, weight/volume of the selective COX-2 inhibitory drug. In one embodiment, a composition of the invention contains a concentration of the selective COX-2 inhibitory drug that is therapeutically or prophylactically equivalent to a celecoxib weight/volume concentration of about 0.1% to about 50%, preferably about 0.5% to about 20%, and most preferably about 1% to about 10%. In another embodiment, a composition of the invention has relatively high loading of the drug and is suitable for a relatively long residence time in a treated eye. In this embodiment the weight/volume concentration of the drug in the composition is about 1.3% to about 50%, preferably about 1.5% to about 30%, and most preferably about 2% to about 20%, for example about 2% to about 10%.

Preferably no more than 3 drops, more preferably no more than 2 drops, and most preferably no more than 1 drop, each of about 15 to about 40 μl, preferably about 20 to about 30 μl, for example about 25 μl, should contain the desired dose of the drug for administration to an eye. Administration of a larger volume to the eye risks loss of a significant portion of the applied composition by lacrimation.

Aqueous compositions of the invention have ophthalmically acceptable pH and osmolality.

The term “ophthalmically acceptable” with respect to a formulation, composition or ingredient herein means having no persistent detrimental effect on the treated eye or the functioning thereof, or on the general health of the subject being treated. It will be recognized that transient effects such as minor irritation or a “stinging” sensation are common with topical ophthalmic administration of drugs and the existence of such transient effects is not inconsistent with the formulation, composition or ingredient in question being “ophthalmically acceptable” as herein defined. However, preferred formulations, compositions and ingredients are those that cause no substantial detrimental effect, even of a transient nature.

By contrast with therapeutic and prophylactic methods involving NSAIDs lacking selectivity for inhibition of COX-2, highly effective relief or prevention of COX-2 mediated ophthalmic disorders can be obtained with greatly reduced risk of the side-effects commonly associated with COX-1 inhibition. Thus the method of the present invention is particularly suitable where conventional NSAIDs are contraindicated, for example in patients with peptic ulcers, gastritis, regional enteritis, ulcerative colitis or diverticulitis, patients with a recurrent history of gastrointestinal lesions, patients with gastrointestinal bleeding, coagulation disorders including anemia such as hypothrombinemia, hemophilia and other bleeding problems, or kidney disease, patients prior to surgery, or patients taking anticoagulants.

A particular advantage over conventional NSAIDs for topical application to eyes is the lack of effect on baseline COX-1 mediated physiological functions including wound healing following eye surgery, and intraocular pressure control.

In an aqueous suspension or solution/suspension composition, the selective COX-2 inhibitory drug can be present predominantly in the form of nanoparticles, i.e., solid particles smaller than about 1 pm in their longest dimension. A benefit of this composition is more rapid release of the drug, and therefore more complete release during the residence time of the composition in a treated eye, than occurs with larger particle size. Another benefit is reduced potential for eye irritation by comparison with larger particle size. Reduced eye irritation in turn leads to a reduced tendency for loss of the composition from the treated eye by lacrimation, which is stimulated by such irritation.

In a related composition the drug preferably has a D₉₀ particle size of about 0.01 to about 200 μm, wherein about 25% to 100% by weight of the particles are nanoparticles. “D₉₀” is defined as a linear measure of diameter having a value such that 90% by volume of particles in the composition, in the longest dimension of the particles, are smaller than that diameter. For practical purposes a determination of D₉₀ based on 90% by weight rather than by volume is generally suitable.

In one composition substantially all of the drug particles in the composition are smaller than 1 μm, i.e., the percentage by weight of nanoparticles is 100% or close to 100%. Average particle size of the drug in this embodiment is preferably about 0.1 to about 0.8 μm (about 100 to about 800 nm), more preferably about 0.15 to about 0.6 μm (about 150 to about 600 nm), and most preferably about 0.2 to about 0.4 μm (about 200 to about 400 nm). The selective COX-2 inhibitory drug can be in crystalline or amorphous form in the nanoparticles. Processes for preparing nanoparticles that involve milling or grinding typically provide the drug in crystalline form, whereas processes that involve precipitation from solution typically provide the drug in amorphous form.

Nanoparticles comprising or consisting essentially of a selective COX-2 inhibitory compound of low water solubility can be prepared according to any process previously applied to the preparation of other poorly water soluble drugs in nanoparticulate form. Suitable processes, without restriction, are illustratively and individually disclosed for such other drugs in the references cited immediately below, all incorporated herein by reference.

U.S. Pat. No. 4,826,689 to Violanto & Fischer.

U.S. Pat. No. 5,145,684 to Liversidge et al.

U.S. Pat. No. 5,298,262 to Na & Rajagopalan.

U.S. Pat. No. 5,302,401 to Liversidge et al.

U.S. Pat. No. 5,336,507 to Na & Rajagopalan.

U.S. Pat. No. 5,340,564 to Illig & Sarpotdar.

U.S. Pat. No. 5,346,702 to Na & Rajagopalan.

U.S. Pat. No. 5,352,459 to Hollister et al.

U.S. Pat. No. 5,354,560 to Lovrecich.

U.S. Pat. No. 5,384,124 to Courteille et al.

U.S. Pat. No. 5,429,824 to June.

U.S. Pat. No. 5,510,118 to Bosch et al.

U.S. Pat. No. 5,518,738 to Eickhoff et al.

U.S. Pat. No. 5,503,723 to Ruddy & Eickhoff.

U.S. Pat. No. 5,534,270 to De Castro.

U.S. Pat. No. 5,536,508 to Canal et al.

U.S. Pat. No. 5,552,160 to Liversidge et al.

U.S. Pat. No. 5,560,931 to Eickhoff et al.

U.S. Pat. No. 5,560,932 to Bagchi et al.

U.S. Pat. No. 5,565,188 to Wong et al.

U.S. Pat. No. 5,569,448 to Wong et al.

U.S. Pat. No. 5,571,536 to Eickhoff et al.

U.S. Pat. No. 5,573,783 to Desieno & Stetsko.

U.S. Pat. No. 5,580,579 to Ruddy et al.

U.S. Pat. No. 5,585,108 to Ruddy et al.

U.S. Pat. No. 5,587,143 to Wong.

U.S. Pat. No. 5,591,456 to Franson & Snyder.

U.S. Pat. No. 5,662,883 to Bagchi et al.

U.S. Pat. No. 5,665,331 to Bagchi et al.

U.S. Pat. No. 5,718,919 to Ruddy & Roberts.

U.S. Pat. No. 5,747,001 to Wiedmann et al.

International Publication No. WO 93/25190.

International Publication No. WO 96/24336.

International Publication No. WO 98/35666.

One of ordinary skill in the art will readily adapt the processes therein described to the preparation of a poorly water soluble selective COX-2 inhibitory drug, for example celecoxib, deracoxib, valdecoxib, rofecoxib, 5-chloro-3-(4-methylsulfonyl)phenyl-2-(2-methyl-5-pyridinyl)pyridine and 2-(3,5-difluorophenyl)-3-[4-(methylsulfonyl)phenyl]-2-cyclopenten-1-one, in nanoparticulate form.

The ophthalmic composition can be an aqueous suspension of a selective COX-2 inhibitory drug of low water solubility, wherein preferably the drug is present predominantly or substantially entirely in nanoparticulate form. Without being bound by theory, it is believed that release of the drug from nanoparticles is significantly faster than from a typical “micronized” composition having a D₉₀ particle size of, for example, about 10 μm or greater.

An aqueous suspension composition of the invention can comprise a first portion of the drug in nanoparticulate form, to promote relatively rapid release, and a second portion of the drug having a D₉₀ particle size of about 10 μm or greater, that can provide a depot or reservoir of the drug in the treated eye for release over a period of time, for example about 2 to about 24 hours, more typically about 2 to about 12 hours, to promote sustained therapeutic effect and permit a reduced frequency of administration.

An aqueous suspension can contain one or more polymers as suspending agents. Useful polymers include water-soluble polymers such as cellulosic polymers, e.g., hydroxypropyl methylcellulose, and water-insoluble polymers such as cross-linked carboxyl-containing polymers.

The composition can be an in situ gellable aqueous solution, suspension or solution/suspension having excipients substantially as disclosed in U.S. Pat. No. 5,192,535, comprising about 0.1% to about 6.5%, preferably about 0.5% to about 4.5%, by weight, based on the total weight of the composition, of one or more cross-linked carboxyl-containing polymers. Such an aqueous suspension is preferably sterile and has an osmolality of about 10 to about 400 mOsM, preferably about 100 to about 250 mOsM, a pH of about 3 to about 6.5, preferably about 4 to about 6, and an initial viscosity, when administered to the eye, of about 1000 to about 30,000 cPs, as measured at 25° C. using a Brookfield Digital LVT viscometer with #25 spindle and 13R small sample adapter at 12 rpm. More typically the initial viscosity is about 5000 to about 20,000 cPs. The polymer component has an average particle size not greater than about 50 μm, preferably not greater than about 30 pm, more preferably not greater than about 20 μm, and most preferably about 1 μm to about 5 μm, in equivalent spherical diameter, and is lightly cross-linked to a degree such that, upon contact with tear fluid in the eye, which has a typical pH of about 7.2 to about 7.4, the viscosity of the suspension rapidly increases, to form a gel. This formation of a gel enables the composition to remain in the eye for a prolonged period without loss by lacrimal drainage.

Preferred carboxyl-containing polymers for use in this composition are prepared from one or more carboxyl-containing monoethylenically unsaturated monomers such as acrylic, methacrylic, ethacrylic, crotonic, angelic, tiglic, α-butylcrotonic, α-phenylacrylic, α-benzylacrylic, α-cyclohexylacrylic, cinnamic, coumaric and umbellic acids, most preferably acrylic acid. The polymers are cross-linked by using less than about 5%, preferably about 0.1% to about 5%, more preferably about 0.2% to about 1%, by weight of one or more polyfunctional cross-linking agents such as non-polyalkenyl polyether difunctional cross-linking monomers, e.g., divinyl glycol. Other suitable cross-linking agents illustratively include 2,3-dihydroxyhexa-1,5-diene, 2,5-dimethylhexa-1,5-diene, divinylbenzene, N,N-diallylacrylamide and N,N-diallylmethacrylamide. Divinyl glycol is preferred. Polyacrylic acid cross-linked with divinyl glycol is called polycarbophil. A polymer system containing polycarbophil is commercially available under the trademark DuraSite® of InSite Vision Inc., Alameda, Calif., as a sustained-release topical ophthalmic delivery system.

This composition can be prepared by a procedure substantially as disclosed in U.S. Pat. No. 5,192,535. One of skill in the art will readily modify such procedure as appropriate for incorporation of a selective COX-2 inhibitory drug in accordance with the present invention.

In another particular formulation, the composition can be an in situ gellable aqueous solution, suspension or solution/suspension having excipients substantially as disclosed in U.S. Pat. No. 4,861,760, comprising about 0.1% to about 2% by weight of a polysaccharide that gels when it contacts an aqueous medium having the ionic strength of tear fluid. A preferred such polysaccharide is gellan gum. This composition can be prepared by a procedure substantially as disclosed in U.S. Pat. No. 4,861,760. One of skill in the art will readily modify such procedure as appropriate for incorporation of a selective COX-2 inhibitory drug in accordance with the present invention.

In another particular formulation, the composition can be an in situ gellable aqueous solution, suspension or solution/suspension having excipients substantially as disclosed in U.S. Pat. No. 5,587,175, comprising about 0.2% to about 3%, preferably about 0.5% to about 1%, by weight of a gelling polysaccharide, preferably selected from gellan gum, alginate gum and chitosan, and about 1% to about 50% of a water-soluble film-forming polymer, preferably selected from alkylcelluloses (e.g., methylcellulose, ethylcellulose), hydroxyalkylcelluloses (e.g., hydroxyethylcellulose, hydroxypropyl methylcellulose), hyaluronic acid and salts thereof, chondroitin sulfate and salts thereof, polymers of acrylamide, acrylic acid and polycyanoacrylates, polymers of methyl methacrylate and 2-hydroxyethyl methacrylate, polydextrose, cyclodextrins, polydextrin, maltodextrin, dextran, polydextrose, gelatin, collagen, natural gums (e.g., xanthan, locust bean, acacia, tragacanth and carrageenan gums and agar), polygalacturonic acid derivatives (e.g., pectin), polyvinyl alcohol, polyvinylpyrrolidone and polyethylene glycol. The composition can optionally contain a gel-promoting counterion such as calcium in latent form, for example encapsulated in gelatin. This composition can be prepared by a procedure substantially as disclosed in U.S. Pat. No. 5,587,175. One of skill in the art will readily modify such procedure as appropriate for incorporation of a selective COX-2 inhibitory drug in accordance with the present invention.

In another particular formulation, the composition can be an in situ gellable aqueous solution, suspension or solution/suspension having excipients substantially as disclosed in European Patent No. 0 424 043, comprising about 0.1% to about 5% of a carrageenan gum. Carrageenans are sulfated polysaccharides; in this embodiment a carrageenan having no more than 2 sulfate groups per repeating disaccharide unit is preferred, including kappa-carrageenan, having 18-25% ester sulfate by weight, iota-carrageenan, having 25-34% ester sulfate by weight, and mixtures thereof. This composition can be prepared by a procedure substantially as disclosed in European Patent No. 0 424 043. One of skill in the art will readily modify such procedure as appropriate for incorporation of a selective COX-2 inhibitory drug in accordance with the present invention.

In another particular formulation, the composition comprises an ophthalmically acceptable mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

In another composition, the selective COX-2 inhibitory drug is solubilized at least in part by an ophthalmically acceptable solubilizing agent. The term “solubilizing agent” herein includes agents that result in formation of a micellar solution or a true solution of the drug. Certain ophthalmically acceptable nonionic surfactants, for example polysorbate 80, can be useful as solubilizing agents, as can ophthalmically acceptable glycols, polyglycols, e.g., polyethylene glycol 400, and glycol ethers.

A class of solubilizing agents having particular utility in solution and solution/suspension compositions of the invention is the cyclodextrins. Suitable cyclodextrins can be selected from α-cyclodextrin, β-cyclodextrin, γ-cyclodextrin, alkylcyclodextrins (e.g., methyl-β-cyclodextrin, dimethyl-β-cyclodextrin, diethyl-β-cyclodextrin), hydroxyalkylcyclodextrins (e.g., hydroxyethyl-β-cyclodextrin, hydroxypropyl-β-cyclodextrin), carboxyalkylcyclodextrins (e.g., carboxymethyl-β-cyclodextrin), sulfoalkylether cyclodextrins (e.g., sulfobutylether-β-cyclodextrin), and the like. Ophthalmic applications of cyclodextrins have been reviewed by Rajewski & Stella (1996), Journal of Pharmaceutical Sciences, 85, 1154, at pages 1155-1159. If desired, complexation of a selective COX-2 inhibitory drug by a cyclodextrin can be increased by addition of a water-soluble polymer such as carboxymethylcellulose, hydroxypropyl methylcellulose or polyvinylpyrrolidone, as described by Loftsson (1998), Pharmazie, 53, 733-740.

One or more ophthalmically acceptable pH adjusting agents or buffering agents can be included in a composition of the invention, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an ophthalmically acceptable range.

One or more ophthalmically acceptable salts can be included in the composition in an amount required to bring osmolality of the composition into an ophthalmically acceptable range. Such salts include those having sodium, potassium or ammonium cations and chloride, citrate, ascorbate, borate, phosphate, bicarbonate, sulfate, thiosulfate or bisulfite anions; preferred salts include sodium chloride, potassium chloride, sodium thiosulfate, sodium bisulfite and ammonium sulfate, with sodium chloride being especially preferred.

Optionally one or more ophthalmically acceptable acids having at least two dissociable hydrogen groups can be included in a polymer-containing composition as interactive agents to retard release of the drug through inhibition of erosion of the polymer, as disclosed in International Patent Publication No. WO 95/03784. Acids useful as interactive agents include boric, lactic, orthophosphoric, citric, oxalic, succinic, tartaric and formic glycerophosphoric acids.

Optionally an ophthalmically acceptable xanthine derivative such as caffeine, theobromine or theophylline can be included in the composition, substantially as disclosed in U.S. Pat. No. 4,559,343, to reduce ocular discomfort associated with administration of the composition.

Optionally one or more ophthalmically acceptable preservatives can be included in the composition to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Optionally one or more ophthalmically acceptable surfactants, preferably nonionic surfactants, can be included in the composition to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Optionally one or more antioxidants can be included in the composition to enhance chemical stability where required. Suitable antioxidants include ascorbic acid and sodium metabisulfite.

One or more ophthalmic lubricating agents can optionally be included in the composition to promote lacrimation or as a “dry eye” medication. Such agents include polyvinyl alcohol, methylcellulose, hydroxypropyl methylcellulose, polyvinylpyrrolidone, etc.

Aqueous suspension compositions of the invention can be packaged in single-dose non-reclosable containers. Such containers can maintain the composition in a sterile condition and thereby eliminate need for preservatives such as mercury-containing preservatives, which can sometimes cause irritation and sensitization of the eye. Alternatively, multiple-dose reclosable containers can be used, in which case it is preferred to include a preservative in the composition.

Topical administration can also involve the use of transdermal administration such as transdermal patches, iontophoresis, electroosmosis or electroporation.

The amount of active ingredient that can be combined with the carrier materials to produce a single dosage form varies depending upon the mammalian host treated and the particular mode of administration.

Treatment Regimen

The dosage regimen to prevent, give relief from, or ameliorate a disease condition mediated by COX-2 or to protect against or treat a further COX-2 related disorder with the compounds or compositions of the present invention is selected in accordance with a variety of factors. These include the type, age, weight, diet, and medical condition of the patient, the severity of the disease, the route of administration, pharmacological considerations such as the activity, efficacy, pharmacokinetics and toxicology profiles of the particular compound employed, whether a drug delivery system is utilized, and whether the compound is administered as part of a drug combination. Thus, the dosage regimen actually employed may vary widely and therefore deviate from the preferred dosage regimen set forth above.

Patients undergoing treatment with the compounds or compositions disclosed herein can be routinely monitored to determine the effectiveness of the therapy. Continuous analysis of such data permits modification of the treatment regimen during therapy so that the optimal effective amount of a therapeutic compound is administered at any point in time, and so that the duration of treatment can be determined as well. In this way, the treatment regimen/dosing schedule can be rationally modified over the course of therapy so that the lowest amount of the therapeutic compound that exhibits satisfactory effectiveness is administered, and so that administration is continued only so long as is necessary to successfully treat the COX-2 related ocular condition.

One advantage of using selective COX-2 inhibitors for the treatment of ocular inflammation and pain is that only the production of inflammatory prostaglandins will be affected. The constitutive COX-1 derived prostaglandin formation that is required for normal physiological function of the eye will not be affected. Another advantage of the use of selective COX-2 inhibitors is that their reduced systemic side effects make their oral use more acceptable, even for the treatment of localized ocular COX-2 mediated conditions. Even in the case where various combinations of therapeutic agents with the COX-2 inhibitor may be required, the use of the COX-2 inhibitor may lower the amount of the other agent required and so reduce potential side effects.

One of the several embodiments of the present invention provides a therapeutic method comprising the use of a COX-2 inhibitor in the prophylaxis of COX-2 mediated ocular disorders. For example one of the many embodiments of the present invention is a method comprising a therapeutic dosage of celecoxib for the prevention of cystoid macular edema.

Combinations

The administration of the present invention may be for either prevention or treatment purposes. The methods and compositions used herein may be used alone or in combination with additional therapies known to those skilled in the art of the prevention or treatment of ocular disorders. By way of example, the COX-2 inhibitor may be administered alone or in combination with other agents, drugs or nutrients.

There are large numbers of agents available for treatment of ocular disorders available in commercial use, in clinical evaluation and in pre-clinical development, which could be selected for use with a COX-2 selective inhibitor for the treatment and prevention of ocular COX-2 mediated disorders by combination drug therapy.

The methods and combinations of the present invention provide one or more benefits. Combinations of COX-2 inhibitors with other compounds, compositions, agents and therapies are useful in treating and preventing ocular COX-2 mediated disorders. Preferably, these combinations are administered at a low dose, that is, at a dose lower than has been conventionally used in clinical situations.

The combinations of the present invention will have a number of uses. For example, through dosage adjustment and medical monitoring, the individual dosages of the therapeutic compounds used in the combinations of the present invention will be lower than are typical for dosages of the therapeutic compounds when used in monotherapy. The dosage lowering will provide advantages including reduction of side effects of the individual therapeutic compounds when compared to the monotherapy. In addition, fewer side effects of the combination therapy compared with the monotherapies will lead to greater patient compliance with therapy regimens.

Alternatively, the methods and combination of the present invention can also maximize the therapeutic effect at higher doses.

When administered as a combination, the therapeutic agents can be formulated as separate compositions that are given at the same time or different times, or the therapeutic agents can be given as a single composition.

Compositions of the invention can be used in co-therapy with one or more drugs other than selective COX-2 inhibitory drugs. Such drugs other than COX-2 inhibitory drugs can be co-administered topically to the eye together with a composition of the invention. A composition of the invention can itself further comprise, in co-formulation with a first drug that is a selective COX-2 inhibitory drug as described herein, a therapeutically or prophylactically effective amount of a second drug that is other than a selective COX-2 inhibitory drug. This second drug can cooperate with the first drug in treating or preventing a COX-2 mediated ophthalmic condition, or it can be used to treat a related or unrelated condition simultaneously affecting the eye.

Any drug having utility as a topical ophthalmic application can be used in co-therapy, co-administration or co-formulation with a composition of the invention as described immediately above. Such drugs include without limitation demulcents; antibiotics, antivirals and other anti-infectives; steroids, NSAIDs and other anti-inflammatory agents; acetylcholine blocking agents; antiglaucoma agents including beta-adrenergic receptor blocking agents, cholinergic agents, sympathomimetics, carbonic anhydrase inhibitors and prostaglandins; antihypertensives; antihistamines; anticataract agents; and topical and regional anesthetics. Illustrative specific drugs include acebutolol, aceclidine, acetazolamide, acetylsalicylic acid (aspirin), N⁴-acetylsulfisoxazole, alclofenac, alprenolol, amfenac, amiloride, aminocaproic acid, β-aminoclonidine, aminozolamide, anisindione, apafant, atenolol, azithromycin, bacitracin, benoxaprofen, benoxinate, benzofenac, bepafant, betamethasone, betaxolol, bethanechol, brimonidine, brinzolamide, bromfenac, bromhexine, bucloxic acid, bupivacaine, butibufen, carbachol, carprofen, carteolol, cephalexin, chloramphenicol, chlordiazepoxide, chlorprocaine, chlorpropamide, chlortetracycline, cicloprofen, cinmetacin, ciprofloxacin, clidanac, clindamycin, clonidine, clonixin, clopirac, cocaine, cromolyn, cyclopentolate, cyproheptadine, demecarium, dexamethasone, dibucaine, diclofenac, diflusinal, dipivefrin, dorzolamide, enoxacin, eperezolid, epinephrine, erythromycin, eserine, estradiol, ethacrynic acid, etidocaine, etodolac, fenbufen, fenclofenac, fenclorac, fenoprofen, fentiazac, flufenamic acid, flufenisal, flunoxaprofen, fluorocinolone, fluorometholone, flurbiprofen and esters thereof, fluticasone propionate, furaprofen, furobufen, furofenac, furosemide, gancyclovir, gentamycin, gramicidin, hexylcaine, homatropine, hydrocortisone, ibufenac, ibuprofen and esters thereof, idoxuridine, indomethacin, indoprofen, P¹,P⁴-di(uridine-5′-)tetraphosphate (INS-365), interferons, isobutylmethylxanthine, isofluorophate, isopropyl unoprostone, isoproterenol, isoxepac, ketoprofen, ketorolac, labetolol, lactorolac, latanoprost, levo-bunolol, lidocaine, linezolid, lonazolac, loteprednol, (9S)-9-[(dimethylamino)methyl]-6,7,10,11-tetrahydro-9H,18H-5,21:12,17-dimethenodibenzo[e,k]pyrrolo[3,4-h][1,4,13]oxadiazacyclohexadecine-18,20(19H)-dione (LY-333531), meclofenamate, medrysone, mefenamic acid, mepivacaine, metaproterenol, methanamine, methylprednisolone, metiazinic, metipranolol, metoprolol, metronidazole, minopafant, miroprofen, modipafant, nabumetome, nadolol, namoxyrate, naphazoline, naproxen and esters thereof, neomycin, nepafenac, nitroglycerin, norepinephrine, norfloxacin, nupafant, olfloxacin, olopatadine, oxaprozin, oxepinac, oxyphenbutazone, oxyprenolol, oxytetracycline, penicillins, perfloxacin, phenacetin, phenazopyridine, pheniramine, phenylbutazone, phenylephrine, phenylpropanolamine, phospholine, pilocarpine, pindolol, pirazolac, piroxicam, pirprofen, polymyxin, polymyxin B, prednisolone, prilocaine, prinomastat, probenecid, procaine, proparacaine, protizinic acid, rimexolone, salbutamol, scopolamine, sotalol, sulfacetamide, sulfanilic acid, sulindac, suprofen, tenoxicam, terbutaline, tetracaine, tetracycline, theophyllamine, timolol, tobramycin, tolmetin, travoprost, triamcinolone, trimethoprim, trospectomycin, unoprostol, vancomycin, vidarabine, vitamin A, warfarin, zomepirac and pharmaceutically acceptable salts thereof.

In an especially preferred combination, a composition of the invention is administered in co-therapy or co-formulation with a prostaglandin such as latanoprost, travoprost or isopropyl unoprostone.

Compositions of the present invention can be prepared by methods known in the art and described in patents and publications cited herein and incorporated herein by reference.

Biological Assays

The utility of the present invention can be shown by the following assays. These assays are performed in vitro and in animal models essentially using procedures recognized to show the utility of the present invention.

Rat Endotoxin-Induced-Uveitis Test

The rat endotoxin-induced-uveitis test is performed with materials, reagents and procedures essentially as described by Tsuji, et al. (Exp. Eye Res., 64, 31 (1997)). Female six-seven week old Lewis rats weighing about 160 g are housed under a 12 hr light-dark cycle with humidity maintained at 55% and room temperature at 23° C. Food and water are available ad libitum. The animals are given subcutaneous injection in the footpads of lipopolysaccharide (LPS) endotoxin (500 μg per kg dissolved in saline at a concentration of 1 mg/mL) from Salmonella typhimurium to induce uveitis.

In topical applications, the test COX-2 inhibitors (0.01-1.0%) are instilled (5 μl/eye) three times at 1 hr before and 3 and 7 hrs after injection of LPS. For systemic application, the test COX-2 inhibitor is injected subcutaneously 3 hr after LPS injection.

Twelve hours after injection of LPS, the animals are killed and both eyes of each animal are used. The aqueous humor is collected by puncturing the anterior chamber of the eye using a 27 gauge needle. The aqueous humor samples (5 μl) are placed into phosphate-buffered saline (495 μl) containing 1% paraformaldehyde. A flow cytometry system is used to count the cell number in the aqueous humor. The average cell number for both eyes of each animal is used for the statistical analysis of results.

Guinea Pig Endotoxin-Induced-Uveitis Test

The guinea pig endotoxin-induced-uveitis test is performed with materials, reagents and procedures essentially as described by Tsuji, et al. (Inflamm. Res., 46, 486 (1997)). Male five-six week old Hartley guinea pigs weighing 300-450 g are housed under a 12 hr light-dark cycle with humidity being maintained at 55% and room temperature at 23° C. Food and water are available ad libitum. Lipopolysaccharide (LPS) from E. coli (10 ul) is injected intracamerally using a 30 gauge needle into each eye of the guinea pigs under pentobarbital anesthesia. Paracentesis accompanies this procedure. In topical applications, the test COX-2 inhibitors (0.01-1.0%) are instilled (10 μl/eye) two times at 1 hr before and 3 hrs after injection of LPS.

Twelve hours after injection of LPS, the animals are sacrificed by exsanguination and both eyes of each animal are used. The aqueous humor is collected by puncturing the anterior chamber of the eye using a 27 gauge needle. The aqueous humor samples (5 μl) are placed into phosphate-buffered saline (495 μl) containing 1% paraformaldehyde. A flow cytometry system is used to count the cell number in the aqueous humor. The average cell number for both eyes of each animal is used for the statistical analysis of results.

A similar LPS induced uveitis model in the rabbit may be performed with materials, reagents and procedures essentially as described by Howes, et al. (Journal of Ocular Pharmacology, 10, 289 (1994)).

Trauma-Induced Rabbit Ocular Inflammation Test

The trauma-induced rabbit ocular inflammation test is performed with materials, reagents and procedures essentially as described by Gamache, et al. (Inflammation, 24, 357 (2000)). New Zealand Albino rabbits (2-2.5 kg) are treated with a single topical ocular dose of the test COX-2 inhibitors (0.01-1.0 %) or vehicle (50 μl/eye), administered bilaterally. At various intervals after dosing (15 min to 8 hrs), each eye is treated with one drop (5 μL) of 0.5% proparacaine, and within 5 min, trauma is induced by paracentesis. Aqueous humor (150 μl/eye) is removed by puncture of the cornea with a 27 g needle. One hundred microliters of aqueous humor is diluted with 100 μl of EDTA in saline (2%, pH 7.4) and stored at −70° C. for later analysis of protein and PGE₂ content. Animals are sacrificed with an overdose of sodium pentobarbital (100 mg/kg) in the marginal ear vein thirty minutes after the initial paracentesis. Post-trauma aqueous humor samples are obtained and stored as described above.

The protein concentration of the aqueous humor samples is assayed according to the calorimetric method essentially as described by Bradford, et al. (Anal. Biochem., 72, 248 (1976)). To monitor formation of PGE₂ by radio-HPLC, the aqueous humor extracts are incubated with 10 μM of [1-¹⁴C]-labeled arachidonic acid (10 μCi/mol) for 10 min at 37° C. PGE₂ is quantified in organic extracts by HPLC essentially as described by Powell (Anal. Biochem., 148, 59 (1985)).

Evaluation of COX-1 and COX-2 activity in vitro

The compounds of this invention exhibit in vitro inhibition of COX-2. The COX-2 inhibition activity of the compounds of this invention is determined by the following methods.

a. Preparation of recombinant COX baculoviruses

A 2.0 kb fragment containing the coding region of either human or murine COX-1 or human or murine COX-2 is cloned into a BamHl site of the baculovirus transfer vector pVL1393 (Invitrogen) to generate the baculovirus transfer vectors for COX-1 and COX-2 in a manner similar to the method of D. R. O'Reilly et al. (Baculovirus Expression Vectors: A Laboratory Manual (1992)). Recombinant baculoviruses are isolated by transfecting 4 pg of baculovirus transfer vector DNA into SF9 insect cells (2×10 e8) along with 200 ng of linearized baculovirus plasmid DNA by the calcium phosphate method. See M. D. Summers and G. E. Smith, A Manual of Methods for Baculovirus Vectors and Insect Cell Culture Procedures, Texas Agric. Exp. Station Bull., 1555 (1987). Recombinant viruses are purified by three rounds of plaque purification and high titer (lOE7-lOE8 pfu/ml) stocks of virus are prepared. For large scale production, SF9 insect cells are infected in 10 liter fermentors (0.5×10⁶/ml) with the recombinant baculovirus stock such that the multiplicity of infection is 0.1. After 72 hours the cells are centrifuged and the cell pellet homogenized in Tris/Sucrose (50 mM: 25%, pH 8.0) containing 1% 3-[(3-cholamidopropyl)dimethylammonio]-1-propanesulfonate (CHAPS). The homogenate is centrifuged at 10,000×G for 30 minutes, and the resultant supernatant is stored at −80° C. before being assayed for COX activity.

b. Assay for COX-1 and COX-2 activity

COX activity is assayed as PGE2 formed/μg protein/time using an ELISA to detect the prostaglandin released. CHAPS-solubilized insect cell membranes containing the appropriate COX enzyme are incubated in a potassium phosphate buffer (50 mM, pH 8.0) containing epinephrine, phenol, and heme with the addition of arachidonic acid (10 μM). Compounds are pre-incubated with the enzyme for 10-20 minutes prior to the addition of arachidonic acid. Any reaction between the arachidonic acid and the enzyme is stopped after ten minutes at 37° C./room temperature by transferring 40 μl of reaction mix into 160 μl ELISA buffer and 25 μM indomethacin. The PGE2 formed is measured by standard ELISA technology (Cayman Chemical).

The examples herein can be performed by substituting the generically or specifically described reactants or operating conditions of this invention for those used in the preceding examples.

The invention being thus described, it is apparent that the same can be varied in many ways. Such variations are not to be regarded as a departure from the spirit and scope of the present invention, and all such modifications and equivalents as would be obvious to one skilled in the art are intended to be included within the scope of the following claims.

What is claimed is:

1. A therapeutic method for treating or preventing an ocular COX-2 mediated disorder comprising administering an ocular COX-2 mediated disorder-effective amount of a source of a COX-2 inhibitor compound to a mammal in need of such treatment, wherein the disorder is selected from the group consisting of blepharitis, post-operative inflammation and pain from corneal transplant surgery, endophthalmitis, episcleritis, keratitis, keratoconjunctivitis, keratoconjunctivitis sicca, post-operative inflammation and pain from lens implantation surgery, Mooren's ulcer and post-operative inflammation and pain from retinal detachment surgery.

2. The therapeutic method of claim 1 wherein the source of the COX-2 inhibitor comprises a COX-2 inhibitor.

3. The therapeutic method of claim 2 wherein the COX-2 inhibitor is selected from the group consisting of celecoxib, deracoxib, valdecoxib, a benzopyran COX-2 inhibitor, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methylbutoxy)-5-[4-(methylsulfonyl)phenyl]-3(2H)-pyridazinone.

4. The therapeutic method of claim 3 wherein the COX-2 inhibitor is celecoxib.

5. The therapeutic method of claim 3 wherein the COX-2 inhibitor is deracoxib.

6. The therapeutic method of claim 3 wherein the COX-2 inhibitor is valdecoxib.

7. The therapeutic method of claim 3 wherein the COX-2 inhibitor is a benzopyran COX-2 inhibitor.

8. The therapeutic method of claim 3 wherein the COX-2 inhibitor is rofecoxib.

9. The therapeutic method of claim 3 wherein the COX-2 inhibitor is etoricoxib.

10. The therapeutic method of claim 3 wherein the COX-2 inhibitor is 2-(3,5-difluorophenyl)-3-[4-(methylsulfonyl)phenyl]-2-cyclopenten-1-one.

11. The therapeutic method of claim 3 wherein the COX-2 inhibitor is 2-(3,4-difluorophenyl)-4-(3-hydroxy-3-methylbutoxy)-5-[4-(methylsulfonyl)phenyl]-3 (2H)-pyridazinone.

12. The therapeutic method of claim 1 wherein the source of the COX-2 inhibitor comprises a prodrug of a COX-2 inhibitor.

13. The therapeutic method of claim 12 wherein the prodrug of the COX-2 inhibitor is parecoxib.

14. The therapeutic method of claim 1 wherein the ocular COX-2 mediated disorder is Mooren's ulcer.

15. The therapeutic method of claim 14 wherein the source of the COX-2 inhibitor further comprises one or more ophthalmically acceptable excipient ingredients that reduce the rate of removal of the composition from the eye by lacrimation such that the composition has an effective residence time in the eye of about 2 to about 24 hours.

16. A pharmaceutical composition for treating or preventing Mooren's ulcer, in a mammal in need of such treatment, consisting essentially of a source of a COX-2 inhibitor compound and one or more ophthalmically acceptable excipient ingredients that reduce the rate of removal of the composition from the eye by lacrimation such that the composition has an effective residence time in the eye of about 2 to about 24 hours.

17. A therapeutic method for treating or preventing an ocular COX-2 mediated disorder comprising administering an ocular COX-2 mediated disorder-effective amount of celecoxib to a mammal in need of such treatment, wherein the disorder is selected from the group consisting of macular edema, intraoperative miosis and ocular pain.

18. The therapeutic method of claim 17 wherein the ocular COX-2 mediated disorder is macular edema.

19. A therapeutic method for treating or preventing an ocular COX-2 mediated disorder comprising administering an ocular COX-2 mediated disorder-effective amount of deracoxib to a mammal in need of such treatment, wherein the disorder is selected from the group consisting of post-operative inflammation and pain from cataract surgery, acute injury to the eye tissue, glaucoma, macular edema, intraoperative miosis, ocular pain, photophobia, post-operative inflammation and pain from refractive surgery, retinitis, retinopathies and uveitis.

20. The therapeutic method of claim 19 wherein the ocular COX-2 mediated disorder is post-operative inflammation and pain from cataract surgery.

21. The therapeutic method of claim 20 wherein the ocular COX-2 mediated disorder is macular edema.

22. A therapeutic method for treating or preventing an ocular COX-2 mediated disorder comprising administering an ocular COX-2 mediated disorder-effective amount of valdecoxib to a mammal in need of such treatment, wherein the disorder is selected from the group consisting of macular edema, intraoperative miosis and ocular pain.

23. The therapeutic method of claim 22 wherein the ocular COX-2 mediated disorder is macular edema.

24. A therapeutic method for treating or preventing an ocular COX-2 mediated disorder comprising administering an ocular COX-2 mediated disorder-effective amount of a benzopyran COX-2 inhibitor to a mammal in need of such treatment, wherein the disorder is selected from the group consisting of glaucoma, macular edema, intraoperative miosis and ocular pain.

25. The therapeutic method of claim 24 wherein the ocular COX-2 mediated disorder is macular edema.

26. A therapeutic method for treating or preventing an ocular COX-2 mediated disorder comprising administering an ocular COX-2 mediated disorder-effective amount of parecoxib to a mammal in need of such treatment, wherein the disorder is selected from the group consisting of conjunctivitis, glaucoma, macular edema, intraoperative miosis and ocular pain.

27. The therapeutic method of claim 26 wherein the ocular COX-2 mediated disorder is macular edema.

28. A therapeutic method for treating or preventing an ocular COX-2 mediated disorder comprising administering an ocular COX-2 mediated disorder-effective amount of rofecoxib to a mammal in need of such treatment, wherein the disorder is selected from the group consisting of post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, sarcoidosis and uveitis.

29. The therapeutic method of claim 28 wherein the ocular COX-2 mediated disorder is post-operative inflammation and pain from cataract surgery.

30. The therapeutic method of claim 28 wherein the ocular COX-2 mediated disorder is macular edema.

31. A therapeutic method for treating or preventing an ocular COX-2 mediated disorder comprising administering an ocular COX-2 mediated disorder-effective amount of etoricoxib to a mammal in need of such treatment, wherein the disorder is selected from the group consisting of post-operative inflammation and pain from cataract surgery, conjunctivitis, acute injury to the eye tissue, macular edema, intraoperative miosis, ocular pain, photophobia, post-operative inflammation and pain from refractive surgery, retinitis, retinopathies, sarcoidosis and uveitis.

32. The therapeutic method of claim 31 wherein the ocular COX-2 mediated disorder is post-operative inflammation and pain from cataract surgery.

33. The therapeutic method of claim 31 wherein the ocular COX-2 mediated disorder is macular edema.

34. A therapeutic method for treating or preventing an ocular COX-2 mediated disorder comprising administering an ocular COX-2 mediated disorder-effective amount of 2-(3,5-difluorophenyl)-3-[4-(methylsulfonyl)phenyl]-2-cyclopenten-1-one to a mammal in need of such treatment, wherein the disorder is selected from the group consisting of post-operative inflammation and pain from cataract surgery, conjunctivitis, acute injury to the eye tissue, macular edema, intraoperative miosis, ocular pain, photophobia, post-operative inflammation and pain from refractive surgery, retinitis, sarcoidosis and uveitis.

35. The therapeutic method of claim 34 wherein the ocular COX-2 mediated disorder is post-operative inflammation and pain from cataract surgery.

36. The therapeutic method of claim 34 wherein the ocular COX-2 mediated disorder is macular edema.

37. A therapeutic method for treating or preventing an ocular COX-2 mediated disorder comprising administering an ocular COX-2 mediated disorder-effective amount of 2-(3,4-difluorophenyl)-4-(3-hydroxy-3-methylbutoxy)-5-[4-(methylsulfonyl)phenyl]-3(2H)-pyridazinone to a mammal in need of such treatment, wherein the disorder is selected from the group consisting of post-operative inflammation and pain from cataract surgery, conjunctivitis, acute injury to the eye tissue, glaucoma, macular edema, intraoperative miosis, ocular pain, photophobia, post-operative inflammation and pain from refractive surgery, retinitis, retinopathies, sarcoidosis and uveitis.

38. The therapeutic method of claim 37 wherein the ocular COX-2 mediated disorder is post-operative inflammation and pain from cataract surgery.

39. The therapeutic method of claim 37 wherein the ocular COX-2 mediated disorder is macular edema.
